SEQUENCE LISTING (5 sequences)

SEQ ID NO: 1 - DNA, 414 bp, Homo sapiens
atgaactttctgctgtcttgggtgcattggagccttgccttgctgctctacctccaccatgccaagtggtcccaggctgcacccatggcagaaggaggagggcagaatcatcacgaagtggtgaagttcatggatgtctatcagcgcagctactgccatccaatcgagaccctggtggacatcttccaggagtaccctgatgagatcgagtacatcttcaagccatcctgtgtgcccctgatgcgatgcgggggctgctgcaatgacgagggcctggagtgtgtgcccactgaggagtccaacatcaccatgcagattatgcggatcaaacctcaccaaggccagcacataggagagatgagcttcctacagcacaacaaatgtgaatgcagatgtgacaagccgaggcggtga

SEQ ID NO: 2 - protein, 181 aa, Homo sapiens
MNFLLSWVHWSLALLLYLHHAKWSQAAPMAEGGGQNHHEVVKFMDVYQRSYCHPIETLVDIFQEYPDEIEYIFKPSCVPLMRCGGCCNDEGLECVPTEESNITMQIMRIKPHQGQHIGEMSFLQHNKCECRPCGPCSERRKHLFVQDPQTCKCSCKNTDSRCKARQLELNERTCRCDKPRR

SEQ ID NO: 3 - DNA, 336 bp, Homo sapiens
gcacccatggcagaaggaggagggcagaatcatcacgaagtggtgaagttcatggatgtctatcagcgcagctactgccatccaatcgagaccctggtggacatcttccaggagtaccctgatgagatcgagtacatcttcaagccatcctgtgtgcccctgatgcgatgcgggggctgctgcaatgacgagggcctggagtgtgtgcccactgaggagtccaacatcaccatgcagattatgcggatcaaacctcaccaaggccagcacataggagagatgagcttcctacagcacaacaaatgtgaatgcagatgtgacaagccgaggcggtga

SEQ ID NO: 4 - protein, 155 aa, Homo sapiens
APMAEGGGQNHHEVVKFMDVYQRSYCHPIETLVDIFQEYPDEIEYIFKPSCVPLMRCGGCCNDEGLECVPTEESNITMQIMRIKPHQGQHIGEMSFLQHNKCECRPCGPCSERRKHLFVQDPQTCKCSCKNTDSRCKARQLELNERTCRCDKPRR

SEQ ID NO: 5 - DNA, 647 bp, Homo sapiens
atgaactttctgctgtcttgggtgcattggagccttgccttgctgctctacctccaccatgccaagtggtcccaggctgcacccatggcagaaggaggagggcagaatcatcacgaagtggtgaagttcatggatgtctatcagcgcagctactgccatccaatcgagaccctggtggacatcttccaggagtaccctgatgagatcgagtacatcttcaagccatcctgtgtgcccctgatgcgatgcgggggctgctgcaatgacgagggcctggagtgtgtgcccactgaggagtccaacatcaccatgcagattatgcggatcaaacctcaccaaggccagcacataggagagatgagcttcctacagcacaacaaatgtgaatgcagccaaagaaagatagagcaagacaagaaaaaaaatcagttcgaggaaagggaaaggggcaaaaacgaaagcgcaagaaatcccggtataagtcctggagcgttccctgtgggccttgctcagagcggagaaagcatttgtttgtacaagatccgcagacgtgtaaatgttcctgcaaaaacacagactcgcgttgcaaggcgaggcagcttgagttaaacgaacgtacttgcagatgtgacaagccgaggcggtga

(19) United States
(12) Patent Application Publication
(10) Pub. No.: US 2010/0287625 A1
(43) Pub. Date: Nov. 11, 2010

(54) VEGF VARIANTS

(75) Inventors: Alain Colige, Dave (BE); Pierre Mineur, Verviers (BE); Charles Lambert, Angleur (BE)

Correspondence Address: FENWICK & WEST LLP, SILICON VALLEY CENTER, 801 CALIFORNIA STREET, MOUNTAIN VIEW, CA, 94041, US

(73) Assignee: UNIVERSITE DE LIEGE, Angleur (BE)

(21) Appl. No.: 12/161,470

(22) PCT Filed: Jan. 18, 2007
(86) PCT No.: PCT/IB07/00586

§ 371 Date: Jul. 23, 2010

(30) Foreign Application Priority Data
Jan. 19, 2006 (GB) 0601098.7
Oct. 2, 2006 (EP) 06255087.6

(51) Int. Cl. G01N 33/00; C07K 14/475; C07H 21/04; C12N 15/63; C12N 5/10; C07K 16/22; C12Q 1/68; A61K 38/18; G01N 33/53; A01K 67/00

(52) U.S. Cl. 800/3; 530/350; 536/23.5; 435/320.1; 435/325; 530/387.9; 435/6; 514/8.1; 435/7.1; 800/13

(57) ABSTRACT

This invention relates to a Vascular Endothelial Growth Factor (VEGF) polypeptide, which polypeptide lacks an amino acid sequence encoded by exon 5 of the VEGF gene. This variant of VEGF is capable of eliciting activities associated with VEGF whilst showing resistance to proteolytic degradation. The invention provides uses of this protein and nucleic acid sequences from the encoding genes in the diagnosis, prevention and treatment of disease.

### TECHNICAL FIELD

This invention relates to novel Vascular Endothelial Growth Factor (VEGF) polypeptides and to methods of therapy and diagnosis using these polypeptides.

All documents cited herein are incorporated by reference in their entirety.

### BACKGROUND ART

Vascular endothelial growth factor-A (VEGF-A) is a disulphide-bonded dimeric glycoprotein with a molecular mass of 34 to 45 kDa. It stimulates vascular endothelial cell survival, proliferation, migration and differentiation, alters their pattern of gene expression and delays senescence, thereby promoting angiogenesis, vasculogenesis and lymphangiogenesis [1]. It also causes permeabilization of microvasculature (a potency that explains its former naming as vascular permeability factor), a critical step in tumour development and metastasis. Fine tuning regulation of its expression is essential for development, as both homozygous and heterozygous deletions in mice are embryonic lethals [2,3]. VEGF-A acts through binding to plasma membrane VEGF receptors-1 (VEGFR1, flt-1) and -2 (VEGFR2, KDR/flk-1), activating trans-phosphorylation and downstream signalling cascades. Potentiation of the binding of VEGF-A to VEGF-R2 is exerted by neuropilin-1, a non kinase receptor [4]. Soluble forms of the VEGFR-1 and VEGFR-2 act as decoy receptors that negatively regulate VEGF activity [5].

The VEGF-A gene contains eight exons. Human VEGF mRNA undergoes alternative splicing that generates several isoforms [6-12]. All the mRNA variants described to date contain the sequence of exons 1 to 5 and differ only by the alternative presence of 3′-sequences. Exon 1 and a part of exon 2 code for the signal peptide, and exons 3 and 4 for the motifs involved in binding to the VEGF receptor-1 and receptor-2, respectively [13,14]. Exon 3 also encodes the site of glycosylation of the molecule [15]. No specific function has been assigned to exon 5, but the amino acid sequence that it encodes contains the main cleavage site of the molecule by plasmin [13]. Cleavage by matrix metalloproteinases (MMP), including MMP-3, in the sequence encoded by exon 5 of mouse VEGF was also demonstrated [16].

The peptide sequence encoded by exons 6 and 7 is involved in binding to heparan sulphate proteoglycans, neuropilin-1 and -2 and other, undefined receptors on cells [8].

VEGF function is associated with a number of disorders. There are some, such as cancer, where downregulation of inappropriate VEGF-dependent angiogenesis would be of benefit. There are other applications, such as defective wound healing and ischemic disorders, where an increase in VEGF- dependent angiogenesis would be beneficial. There thus exists a need for the discovery of novel VEGF variants and novel ways of modulating the activity of these polypeptides.

### DISCLOSURE OF THE INVENTION

The inventors have identified and described a new naturally occurring isoform of VEGF mRNA that lacks exon 5, in addition to lacking exons 6 and 7. This is the first description of a VEGF isoform lacking exon 5 in human.

According to a first aspect of the invention, therefore, there is provided a VEGF polypeptide which lacks an amino acid sequence encoded by exon 5 of the VEGF gene. The inventors have shown that a VEGF variant of this type are found naturally, are active in eliciting activities associated with VEGF, and are resistant to proteolytic degradation. A prototypic example of a polypeptide according to the invention is provided by the isoform known herein as VEGF111. This has been named according to conventional nomenclature, where the name given to the polypeptide represents the number of constituent amino acids after cleavage of the signal peptide.

The inventors have demonstrated that VEGF111 is capable of conventional VEGF activity, including phosphorylating VEGF-R2, activating the ERK1/2 signal pathways, inducing transient increases of intracellular free calcium concentration in endothelial cells and increasing the proliferation rate of human umbilical vein endothelial cells (HUVEC) at levels similar to other VEGF isoforms, e.g. VEGF165 and VEGF121. Moreover the inventors have demonstrated that VEGF111 promotes the development of vasculature in mice embryoid bodies and in adult mice. In summary, VEGF111 shows biological activity comparable with previously known VEGF isoforms.

The inventors have also demonstrated that VEGF111, lacking the amino acid sequence encoded by exon 5, is resistant to degradation by plasmin and fluids collected from non-healing wound. Moreover the inventors have demonstrated that treatment by plasmin or fluids collected from non-healing wounds does not affect the activity of VEGF111, while it reduces or suppresses that of VEGF165 and VEGF121. All of the naturally occurring VEGF isoforms tested to date are susceptible to degradation by plasmin [13], the main site of cleavage being identified as Arg110-Ala111, encoded within exon 5. Attempts have been made previously to generate a proteolysis resistant VEGF₁₆₅ isoform [17]. Such proteins, however, suffer from the disadvantage that they are not naturally-occurring and, therefore, may be immunogenic in a human host, so causing unwanted side effects, and reducing efficacy when used as a medicament.

The novel VEGF111 isoform was initially identified in HaCat and MCF-7 cells subsequent to the cells being irradiated with UV-B radiation (270 nm to 350 nm, peak at 310 nm). The expression of VEGF111 progressively increased with the energy of the irradiation. Although the VEGF189, VEGF165 and VEGF121 isoforms were also detected in these cell lines, their levels of expression were not affected or decreased by exposure to UV-B radiation.

Expression of the VEGF111 isoform was also found to be induced by genotoxic pharmacological agents, namely camptothecin, mimosin, and mitomycin C in MCF7 cells. These agents are well known anti-cancer drugs used for the treatment of cancer. This raises the question as to whether anti-cancer therapy might unwittingly induce VEGF111 expression and whether this event might be harmful to patients receiving anti-cancer therapy. The inventors also found that VEGF111 mRNA is induced in blood cells treated with camptothecin ex vivo.

Levels of VEGF111 expression were evaluated in a number of normal adult tissues from human (prostate, breast, brain, lung, cervix, kidney, endometrium and skin) and mice (prostate, breast, brain, lung, cervix, kidney, endometrium, skin, heart, liver, bone, spleen, eye, stomach, muscle, intestine, tendon and placenta), as well as in 6 to 18 day-old mouse embryos. The VEGF111 isoform was not detected in any of these healthy samples, whereas the well known VEGF121, VEGF165 and VEGF189 isoforms were readily detected. This lends support to the contention that VEGF111 expression is potentially causative or reflective of a disease state.

The inventors have also demonstrated that the expression of the human VEGF111 mRNA by UV-B or camptothecin treatment is reduced by siRNA targeting the junction between exon 4 and exon 8. This junction is present in the VEGF111 but absent in all other known isoforms.

The inventors have also demonstrated that the expression of the VEGF111 mRNA induced by UV-B or camptothecin is reduced or suppressed by pharmacological agents affecting the ATM (Ataxia Telengectasia Mutated)/ATR (Ataxia-Related), ERK (Extracellularly Regulated Kinase), p38 MAPK (p38 Mitogen Activated Protein Kinase), JNK/SAPK (Jun N-terminal Kinase/Stress-Activated Protein Kinase), IKK-beta (IκB Kinase-beta) or serine/threonine protein phosphatases.

The invention includes any VEGF polypeptides which lack an amino acid sequence encoded by exon 5 and thus may lack the entire sequence encoded by exon 5 or any portion of exon 5. According to current knowledge, it is the sequence encoded by exons 3 and 4 which is responsible for receptor binding activity. Accordingly, the invention includes VEGF polypeptides which lack the sequence encoded by exon 5 and comprise at least the sequence or partial sequence encoded by exon 4 of the VEGF gene. Such polypeptides may comprise at least the sequence or partial sequence encoded by exons 3 and 4 of the VEGF gene; at least the sequence or partial sequence encoded by exons 3, 4 and 8 of the VEGF gene; at least the sequence or partial sequence encoded by exons 3, 4 and 7 of the VEGF gene; at least the sequence or partial sequence encoded by exons 3, 4 and 6 of the VEGF gene; at least the sequence or partial sequence encoded by exons 3, 4, 7 and 8 of the VEGF gene; at least the sequence or partial sequence encoded by exons 3, 4, 6 and 7 of the VEGF gene; at least the sequence or partial sequence encoded by exons 3, 4, 6 and 8 of the VEGF gene; or at least the sequence or partial sequence encoded by exons 3, 4, 6, 7 and 8 of the VEGF gene. The signal sequence is encoded within exons 1 and 2 of the VEGF gene; accordingly, any one of the above-described polypeptides of the invention may additionally comprise the sequence or partial sequence encoded by exons 1 and/or 2 of the VEGF gene. Polypeptides according to this aspect of the invention may include only the portion of exon 2 that forms part of the mature polypeptide.

In a particular embodiment, there is provided a VEGF polypeptide which comprises the amino acid sequence as recited in SEQ ID NO:2. The invention includes the VEGF111 polypeptide, which consists of the amino acid sequence as recited in SEQ ID NO:2.

The term “VEGFΔ5” as used herein includes any of the VEGF polypeptides described above, which lack an amino acid sequence encoded by exon 5 of the VEGF gene. Such VEGFΔ5 polypeptides may also include variants that possess functional or structural characteristics that are substantially similar to a polypeptide of the present invention, including, for example, fragments and mutants of VEGFΔ5 polypeptides. Such variants will display substantially similar activity compared with VEGFΔ5, but may, for example, have been altered or mutated as required, for example, to enhance or suppress a particular activity. Preferably, a variant of this type may be a polypeptide that displays 50%, 60%, 70%, 80%, 90%, 95%, 98%, 99%, 100% or more VEGF-like activity as compared with VEGFΔ5 in a suitable assay for the measurement of biological activity or function. Such variants may also have been altered or mutated as required, for example, to remove their VEGF-like activity. Preferably, a variant of this type may be a polypeptide that displays 50%, 40%, 30%, 20%, 10%, 5%, 2%, or 0% VEGF-like activity as compared with VEGFΔ5 in a suitable assay for the measurement of biological activity or function. By “VEGF-like activity” is meant the ability to phosphorylate VEGF-R2, activate the ERK1/2 signal pathways, induce transient increases of intracellular free calcium concentration in endothelial cells, increase the proliferation rate of HUVEC and/or induce angiogenesis in animals, including humans, or the ability to bind to soluble VEGFR1 and VEGFR2, at comparable levels to other VEGF isoforms, e.g. VEGF165 and VEGF121.

VEGFΔ5 polypeptides according to the invention are capable of conventional VEGF-like activity, including the ability to phosphorylate VEGF-R2 [18], activate the ERK1/2 signal pathways [19], induce transient increases of intracellular free calcium concentration in endothelial cells [20] and increase cell proliferation, for example as measured in HUVEC cells [21] at comparable levels to other VEGF isoforms, e.g. VEGF165 and VEGF121. Examples of assays that may be used to measure these activities are given in the examples herein and in the journal articles referenced above. Preferably, a VEGFΔ5 polypeptide exhibits at least one, preferably two, three or all four, of the above activities at a level of at least 50% of the activity exhibited by VEGF₁₆₅ and VEGF₁₂₁ under equivalent conditions. Preferably, this activity is at least 60%, at least 70%, at least 80%, at least 90%, at least 100%, at least 110%, at least 120%, at least 150%, at least 200% or more of the activity exhibited by VEGF₁₆₅ and VEGF₁₂₁ under equivalent conditions.

VEGFΔ5 polypeptides according to the invention are resistant to plasmin degradation and degradation by proteases present in body fluids as for example fluids collected from chronic ulcers. Resistance to proteolytic degradation can be measured by incubating the polypeptide with appropriate concentrations of proteolytic enzyme or body fluids as fluids collected from chronic ulcers as described herein. The resulting polypeptide products can then be analyzed by any suitable technique, for example, western blotting. VEGFΔ5 polypeptides according to the invention are preferably more than twice as resistant to proteolytic degradation as VEGF polypeptides that include the proteolytic degradation sites in exon 5.

Variant polypeptides according to the invention may be polypeptides that are homologous to the VEGFΔ5 polypeptides. Two polypeptides are said to be “homologous”, as the term is used herein, if the sequence of one of the polypeptides has a high enough degree of identity to the sequence of the other polypeptide. “Identity” indicates that at any particular position in the aligned sequences, the amino acid residue is identical between the sequences. Degrees of identity can be readily calculated ([22-26]). Percentage identity, as referred to herein, is as determined using BLAST version 2.1.3 using the default parameters specified by the NCBI (the National Center for Biotechnology Information; http://www.ncbi.nlm.nih.gov/) [Blosum 62 matrix; gap open penalty=11 and gap extension penalty=1]. Variant polypeptides therefore include natural biological variants (for example, allelic variants or geographical variations within the species from which the polypeptides are derived) and mutants (such as mutants containing 1, 2, 3, 4, 5, 6, 7, 8, 9, 10 or more amino acid substitutions, insertions or deletions) of the VEGFΔ5 polypeptides. Typically, a variant according to this aspect of the invention will show greater than 90% identity, preferably greater than 95%, 97% or 99% identity with the equivalent wild type VEGF amino acid sequence. For example, where the VEGFΔ5 polypeptide comprises the amino acid sequence encoded for by exons 3, 4 and 8, a variant according to this aspect of the invention will show greater than 90% identity, preferably greater than 95%, 97% or 99% identity with the wild type amino acid sequence encoded by exons 3, 4 and 8.

The VEGFΔ5 polypeptides of the present invention may be in the form of mature proteins or may be a pre-, pro- or prepro-proteins that can be activated by cleavage of the pre-, pro- or prepro-portion to produce active mature polypeptides. In such polypeptides, the pre-, pro- or prepro-sequence may be a leader or secretory sequence or may be a sequence that is employed for purification of the mature polypeptide sequence.

The VEGFΔ5 polypeptides of the first aspect of the invention may form part of a fusion protein. For example, it is often advantageous to include one or more additional amino acid sequences which may contain secretory or leader sequences, pro-sequences, sequences which aid in purification, or sequences that confer higher protein stability, for example during recombinant production, or that renders the polypeptide detectable by imaging technology.

Alternatively or additionally, the mature polypeptide may be fused with another compound, such as a compound to increase the half-life of the polypeptide (for example, polyethylene glycol; WO99/55377).

Suitable fusion partners for VEGFΔ5 polypeptides, that can be comprised in the fusion proteins either at the N- or C-terminus, include: immobilization on solid supports, extracellular domains of membrane-bound protein, immunoglobulin constant regions (Fc regions), multimerization domains, domains of extracellular proteins, signal sequences, export sequences, and sequences allowing purification by affinity chromatography (see, for example [27,28]) or sequence allowing imaging, for example fluorescent polypeptides. Other examples will be clear to those of skill in the art [29]. For instance, a polypeptide according to the invention may further comprise a histidine tag, preferably located at the C-terminal of the polypeptide, generally comprising between 1-10 histidine residues, particularly 6 histidine residues.

Polypeptides of the present invention can be prepared in any suitable manner. Such polypeptides include isolated naturally-occurring polypeptides (for example purified from cell culture), recombinantly-produced polypeptides (including fusion proteins), synthetically-produced polypeptides or polypeptides that are produced by a combination of these methods.

The combined discoveries described above have significant ramifications for the treatment and prevention of VEGF-related disorders. These disorders can be divided into two classes. The first class of VEGF-related disorders are those for which downregulation of VEGF-dependent angiogenesis would be of benefit, whereas the second class of disorders are those for which an increase in VEGF-dependent angiogenesis would be an advantage. Angiogenesis is the physiological process involving the formation of new blood vessels from pre-existing vessels. It is a normal process in growth and development, as well as in wound healing. The term “modulation of angiogenesis” as used herein is intended to encompass both the upregulation and the inhibition of angiogenesis.

If the activity of the VEGFΔ5 polypeptides of the invention is in excess in a particular disease state, several approaches are available. One approach comprises administering to a subject an inhibitor compound (antagonist), along with a pharmaceutically acceptable carrier in an amount effective to inhibit the function of the polypeptide, such as by blocking the binding of ligands, substrates, enzymes, receptors, or by inhibiting any second signal, and thereby alleviating the abnormal condition. Preferably, such antagonists are antibodies or aptamers. In another approach, inactive forms of the polypeptide that retain binding affinity for the ligand, substrate, enzyme, receptor, in question, may be administered. The polypeptide may be administered in the form of fragments that retain the relevant portions of the VEGFΔ5 molecule.

In an alternative approach, expression of the VEGFΔ5 polypeptides can be inhibited using expression blocking techniques, such as the use of antisense nucleic acid molecules, either internally generated or separately administered. Modifications of protein synthesis and secretion can be obtained by designing complementary sequences or antisense molecules (DNA, RNA, or PNA) to block translation of mRNA by preventing the transcript from binding to ribosomes. Such oligonucleotides may be administered or may be generated in situ from expression in vivo.

In addition, expression of the polypeptides the invention may be prevented by specifically increasing the degradation of transcripts lacking exon 5 using ribozymes or siRNA specific to its encoding mRNA sequence. Ribozymes are catalytically active RNAs that can be natural or synthetic [30]. Synthetic ribozymes can be designed to specifically cleave mRNAs at selected positions thereby preventing translation of the mRNAs into functional polypeptide. Ribozymes may be synthesised with a natural ribose phosphate backbone and natural bases, as normally found in RNA molecules.

Alternatively the ribozymes may be synthesised with non-natural backbones, for example, 2′-O-methyl RNA, to provide protection from ribonuclease degradation and may contain modified bases. siRNA are short double stranded RNA involved in eliciting the RNAi (RNA interference) response in mammalian cells. RNAi is a phenomenon where an RNA molecule introduced to a cell or expressed in the cell ultimately causes the degradation of the complementary cellular mRNA, and leads to the knockdown of gene activity. siRNA may be modified to provide protection against ribonuclease, for example by addition of deoxyribonucleotides at their 3′-ends, or to make them cell-permeant, for example after association with a membrane-permeant peptide or cholesterol or other lipids, or to make them fluorescent as known by those skilled in the art. In this context the inventors have shown that introduction of siRNA targeting a sequence astride exon 4 and exon 8 of VEGF gene specifically decreases the expression of VEGF111 mRNA induced by UV-B or camptothecin. Therefore, in a preferred embodiment the invention envisages using siRNA which target a sequence astride exon 4 and exon 8 of the VEGF gene to inhibit the expression of the polypeptides the invention. More preferably, the siRNA have the sequences as disclosed in Table 2 of the present application.

In addition the expression of VEGFΔ5 might be lowered by inhibition of the splicing events involved in the skipping of the exon 5. Such an inhibition may be obtained by treating the cells with chemical agents. In this context the inventors have shown that inhibition of the ATM/ATR, IKK-beta, protein phosphatases, p38 MAP kinase, ERK1/2 kinase and Jun N-terminal Kinase/Stress-Activated Protein Kinase (JNK/SAPK) intracellular pathways reduces the level of VEGF111 induced by UV-B and/or camptothecin. Alternatively such an inhibition may be obtained by interfering with the expression of factors involved in the RNA splicing machinery.

Cancer

Accumulating evidence indicates that progressive tumour growth is dependent on angiogenesis. Most tumours persist in situ for a long period of time (from months to years) in an avascular, quiescent status. In this phase the tumour may contain a few million cells. Angiogenesis, the formation of new vessels, is essential for tumour growth and the development of metastases. To spread, tumours need to be supplied by blood vessels that bring oxygen and nutrients and remove metabolic wastes. Beyond the critical volume of 2 cubic millimetres, oxygen and nutrients have difficulty diffusing to the cells in the centre of the tumour, causing a state of cellular hypoxia. The development of new blood vessels is, therefore, an important process in tumour progression. It favours the transition from hyperplasia to neoplasia i.e. the passage from a state of cellular multiplication to a state of uncontrolled proliferation characteristic of tumour cells. Tumour development evolves though a complex multifactor process that involves interaction of pro-angiogenic and anti-angiogenic signals from tumour, endothelial and stromal cells. The angiogenic activity is reflected in the development of novel microvessels in tumour tissue that is quantified by the intratumoral microvessel density (MVD).

Among several molecules implicated in the angiogenesis of tumour tissue, VEGF appears to be most relevant. There is a significant body of evidence which indicates that VEGF is a key activator of angiogenesis [6, 31] and is overexpressed in a number of tumours. By promoting angiogenesis, VEGF favours the feeding of the tumour cells with oxygen and allows the dissemination of metastasis. Such overexpression is associated with a poor patient prognosis. The vascularization level of a solid tumour is also thought to be an excellent indicator of its metastatic potential.

The inhibition of VEGF-dependent angiogenesis would have significant implications for the treatment of cancer. Anti-VEGF humanised antibodies (Bevacizumab, avastin), are under phase III clinical tests [32-34] and have proved beneficial in the treatment of colorectal, breast and lung cancer. However, severe side-effects such as thrombosis, proteinurea (with occasional nephrotic syndrome), pulmonary embolism, myocardial infarction, gastrointestinal perforation, hemoptisis, epistaxis, wound dehiscence and severe haemorrhages, in some instances resulting in fatality, have been reported [32, 35-38]. Some effects might be related to decreased renewal capacity of endothelial cells in response to trauma and exposure to subendothelial collagen [39]. Also, the safety of avastin in children, and its effect on foetal development are unknown, and its use during pregnancy is not recommanded. In addition pharmacological inhibition of VEGF receptor-2 results in regression of vessels in adult mice, and the survival, differentiation and migration of progenitors of haematopoietic cells is mediated by VEGF, suggesting that inhibition of all the VEGF-A isoforms may be detrimental.

As VEGF111 is not expressed in any of the healthy human tissues tested to date, the inventors deem it unlikely to be necessary for development and health. Therefore a specific therapy that targets VEGF isoforms such as VEGF111, that lack exon 5, may advantageously replace therapies targeting overall VEGF.

As stated above, expression of the VEGF111 isoform was found to be induced by well known chemotherapeutic drugs such as camptothecin, mimosin, and mitomycin C. These agents are commonly used for the treatment of cancer and it thus appears that VEGF111 expression might be induced during chemotherapy. In this context the inventors have shown that camptothecin induced VEGF111 expression in the blood of a healthy donor ex vivo. Accordingly, the invention includes a method of reducing the side-effects of chemotherapy in a patient, comprising administering to the patient a compound that reduces VEGF111 or any other VEGFΔ5 expression or activity. Any compound that reduces VEGF111 or any other VEGFΔ5 expression or activity may be used, including in particular antibodies, aptamers, siRNA and small drug compounds. In particular reduced expression of VEGFΔ5 might be obtained by reducing the skipping of VEGF exon 5 during the process of RNA splicing. In this context the inventors have shown that inhibitors of protein phosphatases reduce the expression of VEGF111 in camptothecin-treated cells by more than 90%. This aspect of the invention also allows potential side-effects of anti-cancer therapy to be monitored, by evaluating the level of VEGF111 or any other VEGFΔ5 that is induced during anti-cancer therapy treatment.

In addition to the applications of VEGF111 or any other VEGFΔ5 in disease therapy, the ability to detect VEGF111 or any other VEGFΔ5 also represents a potential improvement in methods of therapeutic monitoring. Detection of VEGF isoforms known to date has proven to be a poor marker of cancer, as VEGF is naturally expressed in almost all organs under natural conditions. In contrast, specific detection of VEGF111 or any other VEGFΔ5 in body fluids or tissues would allow specific and incontrovertible evidence of a disease state.

Rheumatoid Arthritis

The expansion of the synovial lining of joints in rheumatoid arthritis (RA) and the subsequent invasion by the pannus of underlying cartilage and bone necessitate an increase in the vascular supply to the synovium, to cope with the increased requirement for oxygen and nutrients. Angiogenesis is now recognised as a key event in the formation and maintenance of the pannus in RA. This pannus is highly vascularised, suggesting that targeting blood vessels in RA may be an effective future therapeutic strategy. Disruption of the formation of new blood vessels would not only prevent delivery of nutrients to the inflammatory site, but could also lead to vessel regression and possibly reversal of disease.

VEGF has been shown to a have a central involvement in the angiogenic process in RA. The additional activity of VEGF as a vascular permeability factor may also increase oedema and hence joint swelling in RA. It has been shown that inhibition of VEGF activity in murine collagen-induced arthritis, using a soluble VEGF receptor, reduced disease severity, paw swelling, and joint destruction [40]. The inhibition of angiogenesis, in particular by reducing VEGF expression or by blocking VEGF activity or accessibility to its receptors or by tackling VEGF-induced signalling pathways, appears to be a promising avenue for the future treatment of RA. Again, as VEGF111 is not detected in any of the healthy human tissues tested to date, the inventors deem it unlikely to be necessary for development and health. Therefore a specific therapy that targets VEGF isoforms such as VEGF111, that lack exon 5, might advantageously replace therapies targeting overall VEGF for the treatment of this condition.

Psoriasis

Psoriasis is a chronic skin disease occurring in approximately 3% of the population world-wide. It is characterised by excessive growth of the epidermal keratinocytes, inflammatory cell accumulation and excessive dermal angiogenesis [41]. Histologic studies, including electron microscopy, have clearly established that alterations in the blood vessel formation of the skin are a prominent feature of psoriasis. Uncontrolled angiogenesis, epidermal cell proliferation and localised chronic inflammation result in the formation of a psoriatic plaque. The use of agents that target VEGF dependent angiogenesis represent a novel therapeutic strategy in the treatment of inflammatory diseases. A specific therapy that targets VEGFΔ5 isoforms such as VEGF111, might advantageously replace therapies targeting overall VEGF for the treatment of this condition.

Diseases of the Eye

In the healthy eye, the regulation of angiogenesis is critical for preserving visual clarity. Normal avascular tissues include the cornea, and the aqueous and vitreous fluids. Neovascularization in the eye leads to vision loss and blindness in a number of significant conditions. These include:

Pterygium—Pterygium is a proliferation of fibrovascular tissue on the surface of the eye, associated with ultraviolet light exposure. Within the pterygium are abundantly proliferating blood vessels that promote pannus growth and progression.

Corneal Neovascularization—Invasion of new blood vessels into the normally avascular cornea occurs after infection and injury. Corneal neovascularization may be induced by a number of angiogenic growth factors. Basic fibroblast growth factor (bFGF) is normally sequestered within Descemet's membrane and may be mobilised by injury. Inflammatory cells, such as macrophages and monocytes, also contain various angiogenic growth factors and corneal inflammation is a common stimulus for neovascularization.

Rubeosis Iridis—Neovascularization in the trabecular meshwork of the anterior chamber is observed in diabetes. New blood vessels obstruct aqueous outflow leading to glaucoma. Diffusible angiogenic factors, such as VEGF, are thought to originate from ischemic retinal tissues and promote neovascularization in the anterior chamber.

Retinal Neovascularization—Ischemia is thought to be the primary stimulus for neovascularization in the retina. Local hypoxia leads to upregulation of gene expression for hypoxia inducible factor-1 alpha (HIF-1alpha), which in turn, stimulates production of VEGF. While a number of angiogenic growth factors have been detected in vitreous fluid and retinal tissues, VEGF is regarded as the primary angiogenic factor responsible for retinal neovascularization. VEGF is also known as vascular permeability factor (VPF), and pathological retinal microvessels are leaky. VEGF also serves as a paracrine survival factor for angiogenic endothelial cells. Pericytes, associated with the retinal microvasculature, normally inhibit angiogenesis by secreting activated transforming growth factor-beta (TGF-beta). The loss of pericytes preceding diabetic retinopathy may promote neovascularization by decreasing levels of this endogenous angiogenesis inhibitor.

Choroidal Neovascularization—Angiogenesis originating from the choroidal circulation (subretinal neovascularization) is associated with macular edema and degeneration. The angiogenic growth factors, VEGF and FGF are also associated with this process.

Ocular Tumors—Both primary and metastatic tumors in the eye are dependent upon angiogenesis for growth and progression.

The use of agents that target VEGF dependent angiogenesis represent a novel therapeutic strategy in the treatment of the above diseases. In particular, a specific therapy that targets VEGFΔ5 isoforms such as VEGF111, might advantageously replace therapies targeting overall VEGF for the treatment of this condition.

Induction of Angiogenesis

For treating abnormal conditions related to a defective vascularization and formation of neovessels, several approaches are also available. One approach comprises administering to a subject a therapeutically effective amount of a compound that activates the expression of a polypeptide of the invention to alleviate the abnormal condition. Alternatively, a therapeutic amount of said polypeptide in combination with a suitable pharmaceutical carrier may be administered to stimulate the angiogenesis. For example, combination with a suitable pharmaceutical carrier may involve grafting one or more polypeptides of the invention to biologically active dressings.

Gene therapy may be employed to effect the endogenous production of a polypeptide of the invention by the relevant cells in the subject.

Gene therapy of the present invention can occur in vivo or ex vivo. Ex vivo gene therapy requires the isolation and purification of patient cells, the introduction of a therapeutic gene in said cells and introduction of the genetically altered cells back into the patient. In contrast, in vivo gene therapy does not require isolation and purification of a patient's cells.

The therapeutic gene is typically “packaged” for administration to a patient. Gene delivery vehicles may be non-viral, such as liposomes, or replication-deficient viruses, such as adenovirus as described by Berkner, K. L., [42] or adeno-associated virus (AAV) vectors as described by Muzyczka, N., [43] and U.S. Pat. No. 5,252,479. For example, a nucleic acid molecule encoding a polypeptide of the invention may be engineered for expression in a replication-defective retroviral vector. This expression construct may then be isolated and introduced into a packaging cell transduced with a retroviral plasmid vector containing RNA encoding the polypeptide, such that the packaging cell now produces infectious viral particles containing the gene of interest. These producer cells may be administered to a subject for engineering cells in vivo and expression of the polypeptide in vivo (see Chapter 20, Gene Therapy and other Molecular Genetic-based Therapeutic Approaches, (and references cited therein) in Human Molecular Genetics (1996), T Strachan and A P Read, BIOS Scientific Publishers Ltd).

Another approach is the administration of “naked DNA” in which the therapeutic gene is directly injected into the bloodstream or muscle tissue.

Wound Healing

Angiogenesis occurs during development and represents a physiological response to environmental cues [3]. Neovessel formation also occurs in response to stress (wound repair) [44] and recanalization of thrombi after ischemic events [45]. Among the factors that mediate angiogenesis, VEGF has been the subject of extensive research because of its selective effect on endothelial cells [46].

VEGF165 and VEGF121, but not VEGF111, are susceptible to proteolytic degradation by plasmin and fluids from chronic ulcers. As VEGF111 expression by cells may be induced, albeit under particular conditions, it is unlikely to be recognised by the immune system and is a good candidate for use in the treatment of chronic wounds. Accordingly, the invention provides a method for treating or preventing a chronic wound, comprising exposing the wound to a VEGFΔ5 polypeptide as described above. In one embodiment, VEGFΔ5 polypeptide may be applied to a chronic wound topically.

Chronic Ulcers

The degradation of angiogenic mediators has been suggested to be an underlying cause of chronic wounds. Although the expression of VEGF is elevated in chronic wounds, increased proteolytic activity in this environment results in its degradation. In particular Lauer et al. [47] reported that wound fluids collected from chronic ulcers induce the proteolysis of VEGF165, and that inhibitors of serine proteases, as plasmin, reduces this degradation. VEGF165 mutated at Arg110 and Ala111 (i.e. in the exon 5) is resistant to degradation by plasmin and wound fluids from chronic ulcers but remains biologically active, and was suggested to be used for curing chronic ulcers [17]. However, it is not possible to exclude that such a mutated protein induces the production of antibodies against the mutated epitope.

As VEGF111 is produced by cells, albeit under particular conditions, it is unlikely to be recognised by the immune system and is a good candidate for curing wound ulcers, decubitis sores and other such ailments. Accordingly, the invention provides a method for preventing or treating a chronic ulcer, comprising exposing the ulcer to a VEGFΔ5 polypeptide as described above. In one embodiment, VEGFΔ5 polypeptide may be applied topically to a chronic ulcer or similar wound.

Induction of VEGF111 with UV-B

As described above, the inventors have discovered that the expression of VEGF111 can be induced by exposure to UV-B radiation. By UV-B radiation is meant radiation of wave length between 280 nm to 320 nm, preferably with a peak at 310 nm. This discovery has important ramifications for the treatment of pathologies such as chronic wounds, including chronic ulcers. Accordingly, the invention provides a method for treating such pathologies, in particular chronic ulcers, comprising exposing the tissues to UV-B radiation.

In addition the invention provides a method for treating pathologies, in particular chronic ulcers, by administering cells or tissues to the patients, either by injection or by grafting or by any other mean, after said cells or tissues have been exposed to UV-B radiation. In addition the invention provides a method to prepare media containing VEGFΔ5, preferably in the form of VEGF111, by exposing appropriate cells to UV-B radiation in vitro. Said media could then be administered to patients by, for example, injection or topical application.

Ischemia

Ischemia is a condition in which the blood flow (and thus oxygen) is restricted to a part of the body. Ischemia in the heart muscle is referred to as cardiac ischemia, whereas the same condition in the brain is termed an ischemic stroke. Peripheric ischemia is a common condition that can have serious health consequences such as chronic ulcers and circulation disorders, potentially leading to amputation.

VEGF has been shown to enhance angiogenesis markedly in the ischemic brain and reduce neurological deficits during stroke recovery. Inhibition of VEGF at the acute stage of a stroke may also reduce the blood-brain barrier permeability and the risk of haemorrhagic transformation after focal cerebral ischemia [48].

VEGF has also been shown to enhance angiogenesis in the ischemic heart that occurs in response to myocardial ischemia.

The growth of the collateral circulation (the body's natural response to occluded arteries) is often insufficient to prevent ischemia or myocardial infarction. Supplementation with angiogenic growth factors, in particular VEGFΔ5, more preferably VEGF111, represents a potential pharmacologically method of enhancing the rate and magnitude of collateral circulation development. Accordingly, the invention provides a method of treating or preventing ischaemia, comprising exposing a patient to a VEGFΔ5 polypeptide as described above.

Preeclampsia

Preeclampsia is a disorder that occurs in 4-5% of all pregnancies and is a major cause of maternal and neonatal morbidity and mortality. It is characterized by generalized endothelial dysfunction resulting from a defective vascularization of the placenta and leading to hypertension, glomerular endotheliosis and proteinurea. Although circulating levels of VEGF are elevated in women with preeclampsia, it is now considered that the disease is due to high levels of soluble VEGF-receptor 1 that sequester VEGF [49]. The recently described soluble VEGFR-2 may play a similar role.

In this context the administration of proteolysis-resistant VEGF polypeptides, as VEGF111 or VEGFΔ5 or derived polypeptides able to bind soluble VEGFR-1 and/or soluble VEGFR-2 could has benefit for the patients. Accordingly the invention provides a method of lowering the effects of soluble VEGF receptors in diseases.

Erectile Dysfunction

The major types of vascular problems that can result in erectile dysfunction are arterial insufficiency, inadequate impedance of venous outflow (venous leaks), or a combination of both. With age and underlying diseases, especially atherosclerosis, the amount of blood entering the penis is decreased impeding penile erection. As erectile dysfunction becomes more long-term, treatment becomes more difficult, partly due to an additional component of the disease coming into play, namely ischemia. Prolonged ischemia results in a loss of penile muscle mass and an increase in fibrosis. In this patient group optimal therapeutic strategies should include the use of molecules able to regenerate vascular smooth muscle rather than (or as well as) controlling the level of contractility of the existing musculature. Hence, the development of pro-angiogenic therapies for the treatment of erectile dysfunction may be beneficial to patients with severe disease. Animal studies have identified-insulin-like growth factor (IGF-I) and vascular endothelial growth factor (VEGF) as penile angiogenic growth factors [50].

In all the above contexts the biologically active, proteolysis-resistant VEGF111 or other VEGFΔ5, would confer a significant advantage over the proteolytic-sensitive VEGF isoforms known in the art. Accordingly, the invention provides a method of treating or preventing any disease in which VEGF expression is lowered, comprising exposing a patient to a VEGFΔ5 polypeptide as described above.

In a second aspect, the invention provides a purified nucleic acid molecule which encodes a polypeptide of the first aspect of the invention.

The term “purified nucleic acid molecule” preferably refers to a nucleic acid molecule of the invention that (1) has been separated from at least about 50 percent, preferably at least about 70 percent, at least about 90 percent, or at least about 95 percent of proteins, lipids, carbohydrates, or other materials with which it is naturally found when total nucleic acid is isolated from the source cells; (2) is not linked to all or a portion of a polynucleotide to which the “purified nucleic acid molecule” is linked in nature; (3) is operably linked to a polynucleotide which it is not linked to in nature; or (4) does not occur in nature as part of a larger polynucleotide sequence. In a preferred embodiment, genomic DNA molecules are specifically excluded from the scope of the invention. Preferably, the “purified nucleic acid molecule” consists of cDNA or mRNA only.

Preferred embodiments of this aspect of the invention are nucleic acid molecules that are at least 70% identical over their entire length to a nucleic acid molecule encoding the VEGFΔ5 polypeptides and nucleic acid molecules that are substantially complementary to such nucleic acid molecules. Preferably, a nucleic acid molecule according to this aspect of the invention comprises a region that is at least 80% identical over its entire length to such coding sequences, or is a nucleic acid molecule that is complementary thereto. In this regard, nucleic acid molecules at least 90%, preferably at least 95%, more preferably at least 98%, 99% or more identical over their entire length to the same are particularly preferred. Preferred embodiments in this respect are nucleic acid molecules that encode polypeptides which retain substantially the same biological function or activity as the VEGFΔ5 polypeptides.

Preferably, the purified nucleic acid molecule comprises the nucleic acid sequence as recited in SEQ ID NO:1 (encoding the VEGF111 polypeptide). The invention further provides that the purified nucleic acid molecule consists of the nucleic acid sequence as recited in SEQ ID NO:1 (encoding the VEGF111 polypeptide). These molecules also may have a different sequence which, as a result of the degeneracy of the genetic code, encodes a polypeptide SEQ ID NO:2.

The nucleic acid molecules of the invention can also be engineered, using methods generally known in the art, for a variety of reasons, including modifying the cloning, processing, and/or expression of the gene product (the polypeptide). DNA shuffling by random fragmentation and PCR reassembly of gene fragments and synthetic oligonucleotides are included as techniques which may be used to engineer the nucleotide sequences. Site-directed mutagenesis may be used to insert new restriction sites, alter glycosylation patterns, change codon preference, produce splice variants, introduce mutations, deletions, insertions and so forth.

Gene silencing approaches may also be undertaken to down-regulate endogenous expression of a gene encoding a polypeptide of the invention. RNA interference (RNAi) [51] is one method of sequence specific post-transcriptional gene silencing that may be employed. Short dsRNA oligonucleotides are synthesised in vitro and introduced into a cell or produced by the cells after transfection of adequate vectors as known in the state of the art. The sequence specific binding of these dsRNA oligonucleotides triggers the degradation of target mRNA, reducing or ablating target protein expression.

Efficacy of the gene silencing approaches assessed above may be assessed through the measurement of polypeptide expression (for example, by Western blotting), and at the RNA level using RNA blotting technologies or RT-PCR technologies, including TaqMan-based methodologies.

In a third aspect, the invention provides a purified nucleic acid molecule which hybridizes under high stringency conditions with a nucleic acid molecule of the second aspect of the invention. High stringency hybridisation conditions are defined for example as overnight incubation at 42° C. in a solution comprising 50% formamide, 5×SSC (150 mM NaCl, 15 mM trisodium citrate), 50 mM sodium phosphate (pH7.6), 5× Denhardt's solution, 10% dextran sulphate, and 20 microgram/ml denatured, sheared salmon sperm DNA, followed by washing the filters in 0.1×SSC at approximately 65° C., or other formulations of the hybridization and washing solutions or other protocols as known in the state of the art.

The invention also provides a process for detecting a nucleic acid molecule of the invention, comprising the steps of: (a) contacting a nucleic probe according to the invention with a biological sample under hybridizing conditions to form duplexes; and (b) detecting any such duplexes that are formed. Such methods are useful in the diagnostic applications that are described herein. In particular said nucleic acid molecule can be associated with other nucleic acid molecules for simultaneous detection of various mRNAs or cDNA by DNA array technology.

The nucleic acid molecules of the present invention are also valuable for tissue localisation. Such techniques allow the determination of expression patterns of the VEGFΔ5 polypeptides in tissues by detection of the mRNAs that encode them. These techniques include in situ hybridization techniques and nucleotide amplification techniques, such as PCR. Results from these studies provide an indication of the functions of the polypeptides in the organism.

In a fourth aspect, the invention provides a vector, such as an expression vector, that contains a nucleic acid molecule of the second or third aspect of the invention. The vectors of the present invention comprise nucleic acid molecules of the invention and may be cloning or expression vectors. The polypeptides of the invention may be prepared in recombinant form by expression of their encoding nucleic acid molecules in vectors contained within a host cell. Such expression methods are well known to those of skill in the art and many are described in detail by Sambrook et al. (supra) and Fernandez & Hoeffler [52].

In a fifth aspect, the invention provides a host cell transformed with a vector of the fourth aspect of the invention. The host cells of the invention may be prokaryotic or eukaryotic. Introduction of nucleic acid molecules encoding a polypeptide of the present invention into host cells can be effected by methods described in many standard laboratory manuals, such as Davis et al., [53] and Sambrook et al., (supra). Particularly suitable methods include calcium phosphate transfection, DEAE-dextran mediated transfection, transfection, microinjection, cationic lipid-mediated transfection, electroporation, transduction, scrape loading, ballistic introduction or infection [54,55,72). In eukaryotic cells, expression systems may either be transient (for example, episomal) or permanent (chromosomal integration) according to the needs of the system.

Examples of particularly preferred bacterial host cells include streptococci, staphylococci, E. coli, Streptomyces and Bacillus subtilis cells.

Examples of particularly suitable host cells for fungal expression include yeast cells (for example, S. cerevisiae) and Aspergillus cells.

Mammalian cell lines available as hosts for expression are known in the art and include many immortalised cell lines available from the American Type Culture Collection (ATCC) including, but not limited to, Chinese hamster ovary (CHO), HeLa, baby hamster kidney (BHK), monkey kidney (COS), C127, 3T3, BHK, HEK 293, Bowes melanoma and human hepatocellular carcinoma (for example Hep G2) cells and a number of other cell lines.

In a sixth aspect, the invention provides a ligand which binds specifically to the polypeptides of the first aspect of the invention. Preferably, the ligand inhibits the function of a polypeptide of the first aspect of the invention.

Ligands to a polypeptide according to the invention may come in various forms, including natural or modified substrates, enzymes, receptors, small organic molecules such as small natural or synthetic organic molecules of up to 2000 Da, preferably 800 Da or less, peptidomimetics, inorganic molecules, peptides, polypeptides, antibodies, aptamers, structural or functional mimetics of the aforementioned.

In a preferred aspect of the invention, the ligand which binds specifically to the polypeptides of the invention is an antibody or an aptamer. More particularly the antibody is a monoclonal antibody which is immunospecific for a polypeptide according to the invention, which binds specifically to an epitope which lies at the boundary between the amino acid sequence encoded by exon 4 and that encoded by exon 8 in the VEGF111 polypeptide. Other suitable epitopes will be those that lie at the boundary between the amino acid sequence encoded by exon 4 and by part of exon 8; and at the boundary between the amino acid sequence encoded by exon 4 and exon 6 or part of exon 6; and at the boundary between that encoded by exon 4 and exon 7 or part of exon 7 in the VEGFΔ5 polypeptides.

Antibodies of this type can be generated using polypeptides of the present invention or their immunogenic fragments (comprising at least one antigenic determinant). If polyclonal antibodies are desired, a selected mammal, such as a mouse, rabbit, goat, horse or camel; or non-mammal, such as chicken, may be immunised with a polypeptide of the first aspect of the invention. Monoclonal antibodies to the polypeptides of the first aspect of the invention can also be readily produced by one skilled in the art. The general methodology for making monoclonal antibodies using hybridoma technology is well known [56-58]. In addition antibodies of this type can be generated by phage display technology [59]. Panels of monoclonal antibodies produced against the polypeptides of the first aspect of the invention can be screened for various properties, i.e., for isotype, epitope, affinity, etc.

The term “immunospecific” means that the antibodies or aptamers have substantially greater affinity for the polypeptides of the invention than their affinity for other related VEGF polypeptides. As used herein, the term “antibody” refers to intact molecules as well as to fragments thereof, such as Fab, F(ab′)2 and Fv, which are capable of binding to the antigenic determinant in question. As used herein, the term “aptamer” refers to strands of oligonucleotides (DNA or RNA) that can adopt highly specific three-dimensional conformations. Aptamers are designed to have high binding affinities and specificities towards certain target molecules, including extracellular and intracellular proteins.

By “substantially greater affinity” we mean that there is a measurable increase in the affinity for a polypeptide of the invention as compared with the affinity for known secreted proteins. Preferably, there is a measurable increase in the affinity for a polypeptide of the invention as compared with known VEGF isoforms. Preferably, this measurable increase in affinity is at least 1.5-fold, 2-fold, 5-fold 10-fold, 100-fold, 10³-fold, 10⁴-fold, 10⁵-fold, 10⁶-fold or greater for a polypeptide of the invention than for known VEGF proteins.

The antibody may be modified to make it less immunogenic in an individual, for example by humanisation [60-66]. In a further alternative, the antibody may be a “bispecific” antibody, that is, an antibody having two different antigen binding domains, each domain being directed against a different epitope.

Antibodies generated by the above techniques, whether polyclonal or monoclonal, have additional utility in that they may be employed as reagents in immunoassays, radioimmunoassays (RIA), enzyme-linked immunosorbent assays (ELISA) or protein arrays. In these applications, the antibodies can be labelled with an analytically-detectable reagent such as a radioisotope, a fluorescent molecule or an enzyme.

In a seventh aspect, the invention provides a compound that is effective to alter the expression of a natural gene which encodes a polypeptide of the first aspect of the invention or to regulate the activity of a polypeptide of the first aspect of the invention.

Such compounds may be identified using the assays and screening methods disclosed herein.

A compound of the seventh aspect of the invention may either increase (agonise) or decrease (antagonise) the level of expression of the gene or the activity of the polypeptide.

Importantly, the identification of VEGFΔ5 polypeptides allows for the design of screening methods capable of identifying compounds that might prove effective in the treatment and/or diagnosis of disease. Ligands and compounds according to the sixth and seventh aspects of the invention may be identified using such methods. These methods are included as aspects of the present invention.

Another aspect of this invention resides in the use of a VEGFΔ5 gene or VEGFΔ5 polypeptides as a target for the screening of candidate drug modulators, particularly candidate drugs active against VEGF related disorders.

A further aspect of this invention resides in methods of screening of compounds for therapy of VEGF related disorders, comprising determining the ability of a compound to bind to a VEGFΔ5 gene or polypeptide, or a fragment thereof.

A further aspect of this invention resides in methods of screening of compounds for therapy of VEFG-related disorders, comprising testing for modulation of the activity of a VEFGΔ5 gene or polypeptides, or a fragment thereof.

Antisense Nucleic Acids

A compound which is effective to alter the expression of a natural gene may be an antisense nucleic acid molecule. Such molecules generally range in size from 6 to about 50 nucleotides that are antisense to a gene, RNA or cDNA encoding a VEGFΔ5 polypeptide or a portion thereof. In specific aspects, the oligonucleotide is at least 10 nucleotides, at least 15 nucleotides, at least 100 nucleotides, or at least 200 nucleotides. As used herein, a VEGFΔ5 “antisense” nucleic acid refers to a nucleic acid capable of hybridising by virtue of some sequence complementarity to a portion of an RNA (preferably mRNA) encoding a VEGFΔ5 polypeptide. The antisense nucleic acid may be complementary to a coding and/or non-coding region of an mRNA encoding VEGF. The antisense molecules may be polymers that are nucleic acid mimics, such as PNA, morpholino oligos, and LNA. Other types of antisense molecules include short double-stranded RNAs, known as siRNAs, and short hairpin RNAs, and long dsRNA (>50 bp but usually >500 bp).

Such antisense nucleic acids have utility as compounds that prevent VEGFΔ5 expression, and can be used for tumour regression. The antisense nucleic acids of the invention may be double-stranded or single- stranded oligonucleotides, RNA or DNA or a modification or derivative thereof, and can be directly administered to a cell or produced intracellularly by transcription of exogenous, introduced sequences.

In an eighth aspect, the invention provides a polypeptide of the first aspect of the invention, or a nucleic acid molecule of the second or third aspect of the invention, or a vector of the fourth aspect of the invention, or a host cell of the fifth aspect of the invention, or a ligand of the sixth aspect of the invention, or a compound of the seventh aspect of the invention, for use in therapy, monitoring of therapy, or diagnosis and monitoring of diseases in which VEGF is implicated. As listed in detail above, such diseases include cellular trauma, including ulcers, radiation-induced ulcers, any type of wound healing problems; cell proliferative disorders including myeloproliferative disorders such as leukemia, lymphoma, myelodysplastic syndromes and carcinoma; neoplasm, melanoma, lung, colorectal, breast, pancreas, head and neck and other solid tumours; cardiovascular disorders; neurological disorders; diabetes, in particular diabetic blindness, diabetic kidney disease; age-related macular degeneration; rheumatoid arthritis; psoriasis; cerebral and peripheric ischemia; stroke; coronary artery disease; kidney disorders, hemolytic uremic syndrome; developmental disorders, reproductive disorders in particular erectile dysfunction, endometriosis, preeclampsia; and infections.

In a ninth aspect, the invention provides a method of diagnosing a disease in a patient, comprising assessing the level of expression of a natural mRNA encoding a polypeptide of the first aspect of the invention or the level of expression or activity of a polypeptide of the first aspect of the invention in tissue from said patient and comparing said level of expression or activity to a control level, wherein a level that is different to said control level is indicative of disease. Such a method will preferably be carried out in vitro. Similar methods may be used for monitoring the therapeutic treatment of disease in a patient, wherein altering the level of expression or activity of a polypeptide or nucleic acid molecule over the period of time towards a control level is indicative of regression of disease.

One method for detecting polypeptides of the first aspect of the invention comprises the steps of: (a) contacting a ligand, such as an antibody or an aptamer, of the sixth aspect of the invention with a biological sample under conditions suitable for the formation of a ligand-VEGFΔ5 (e.g. VEGF111) polypeptide complex; and (b) detecting said complex.

A number of different such methods according to the ninth aspect of the invention exist, as the skilled reader will be aware, such as methods of nucleic acid hybridisation with short probes, point mutation analysis, reverse-transcription and polymerase chain reaction (PCR) amplification and methods using antibodies or aptamers to detect aberrant protein levels. Similar methods may be used on a short or long term basis to allow therapeutic treatment of a disease to be monitored in a patient.

Nucleic acid molecules according to the present invention can be used as diagnostic reagents. Detection of a mutated form of the gene characterised by the nucleic acid molecules of the invention which is associated with a dysfunction will provide a diagnostic tool that can add to, or define, a diagnosis of a disease, or susceptibility to a disease, which results from under-expression, over-expression or altered spatial or temporal expression of the gene. Individuals carrying mutations in the gene may be detected at the DNA level by a variety of techniques.

Nucleic acid molecules for diagnosis may be obtained from a subject's cells, such as from blood, urine, saliva, expectorations, tissue biopsy or autopsy material. The genomic DNA may be used directly for detection or may be amplified enzymatically by using PCR, ligase chain reaction (LCR), strand displacement amplification (SDA), or other amplification techniques [67-70] prior to analysis.

In one embodiment, this aspect of the invention provides a method of diagnosing a disease in a patient, comprising assessing the level of expression of a natural mRNA encoding a VEGFΔ5 polypeptide according to the invention and comparing said level of expression to a control level, wherein a level that is different to said control level is indicative of disease. The method may comprise the steps of:

a) contacting a sample of tissue from the patient with a nucleic acid probe under stringent conditions that allow the formation of a hybrid complex between a nucleic acid molecule of the invention and the probe;

b) contacting a control sample with said probe under the same conditions used in step a);

c) and detecting the presence of hybrid complexes in said samples;

wherein detection of levels of the hybrid complex in the patient sample that differ from levels of the hybrid complex in the control sample is indicative of disease.

To aid the detection of nucleic acid molecules in the above-described methods, an amplification step, for example using PCR, may be included.

Diseases may be diagnosed by methods comprising determining, from a sample derived from a subject, an abnormally decreased or increased level of VEGFΔ5 polypeptides or mRNA. Decreased or increased expression can be measured at the RNA level using any of the methods well known in the art for the quantitation of polynucleotides, such as, for example, nucleic acid amplification, for instance PCR, RT-PCR, RNase protection, Northern blotting and other hybridization methods.

The invention also provides kits that are useful in these methods for diagnosing disease. A diagnostic kit of the present invention may comprise: (a) a nucleic acid molecule of the present invention; (b) a polypeptide of the present invention; or (c) a ligand of the present invention.

In one aspect of the invention, a diagnostic kit may comprise a container containing a nucleic acid probe that hybridises under stringent conditions with a nucleic acid molecule according to the invention; an additional container/containers containing primers useful for amplifying the nucleic acid molecule; and instructions for using the probe and primers for facilitating the diagnosis of disease. The kit may further comprise an additional container holding an agent for digesting unhybridised RNA.

To detect polypeptides according to the invention, a diagnostic kit may comprise one or more antibodies or aptamers that bind to a polypeptide according to the invention; and a reagent useful for the detection of a binding reaction between the antibody or aptamer and the polypeptide.

In a tenth aspect, the invention provides a pharmaceutical composition comprising a polypeptide of the first aspect of the invention, or a nucleic acid molecule of the second or third aspect of the invention, or a vector of the fourth aspect of the invention, or a host cell of the fifth aspect of the invention, or a ligand of the sixth aspect of the invention, or a compound of the seventh aspect of the invention, in conjunction with a pharmaceutically-acceptable carrier.

These compositions may be suitable as therapeutic or diagnostic reagents, as vaccines, or as other immunogenic compositions, as outlined in detail below.

Once formulated, the compositions of the invention can be administered directly to the subject. The subjects to be treated can be animals; in particular, human subjects can be treated.

In an eleventh aspect, the present invention provides a polypeptide of the first aspect of the invention, or a nucleic acid molecule of the second or third aspect of the invention, or a vector of the fourth aspect of the invention, or a host cell of the fifth aspect of the invention, or a ligand of the sixth aspect of the invention, or a compound of the seventh aspect of the invention, for use in the manufacture of a medicament for the diagnosis or treatment of a disease, including, but not limited to, cellular trauma, including ulcers, radiation-induced ulcers, any type of wound healing problems; cell proliferative disorders including myeloproliferative disorders such as leukemia, lymphoma, myelodysplastic syndromes and carcinoma; neoplasm, melanoma, lung, colorectal, breast, pancreas, head and neck and other solid tumours; cardiovascular disorders; neurological disorders; diabetes, in particular diabetic blindness, diabetic kidney disease; age-related macular degeneration; rheumatoid arthritis; psoriasis; cerebral and peripheric ischemia; stroke; coronary artery disease; kidney disorders, hemolytic uremic syndrome; developmental disorders, reproductive disorders in particular erectile dysfunction, endometriosis, preeclampsia; and infections.

In a twelfth aspect, the invention provides a method of treating a disease in a patient comprising administering to the patient a polypeptide of the first aspect of the invention, or a nucleic acid molecule of the second or third aspect of the invention, or a vector of the fourth aspect of the invention, or a host cell of the fifth aspect of the invention, or a ligand of the sixth aspect of the invention, or a compound of the seventh aspect of the invention.

For diseases in which an increased vascularization and formation of neovessels is desirable the polypeptide, nucleic acid molecule, ligand, cells or compound administered to the patient may be an agonist. Conversely, for diseases in which the expression of the natural gene or activity of the polypeptide is higher in a diseased patient when compared to the level of expression or activity in a healthy patient, the polypeptide, nucleic acid molecule, ligand or compound administered to the patient may be an antagonist. Examples of such antagonists include antisense nucleic acid molecules, ribozymes, ligands, such as antibodies, and agents able to reduce the skipping of VEGF exon 5.

In a thirteenth aspect, the invention provides transgenic or knockout non-human animal that have been transformed to express higher levels of a polypeptide of the first aspect of the invention or to hinder the induction of said polypeptide . Such transgenic animals are very useful models for the study of disease and may also be used in screening regimes for the identification of compounds that are effective in the treatment or diagnosis of such a disease.

Transgenic animals may be created by local modification of somatic cells, or by germ line therapy to incorporate heritable modifications. Such transgenic animals may be particularly useful in the generation of animal models for drug molecules effective as modulators of the polypeptides of the present invention.

The present invention also provides methods for the screening of drug candidates or leads. These screening methods include binding assays and/or functional assays, and may be performed in vitro, in cell systems or in animals.

In this regard, a particular object of this invention resides in the use of a VEGFΔ5 polypeptide as a target for screening candidate drugs for treating or preventing VEGF related disorders.

Another object of this invention resides in methods of selecting biologically active compounds, said methods comprising contacting a candidate compound with a VEGFΔ5 encoding RNA or polypeptide, and selecting compounds that bind to said RNA or polypeptide, either selectively and/or with high affinity. Such a method may in an alternative comprise contacting a candidate compound with a recombinant host cell expressing a VEGFΔ5 polypeptide, and selecting compounds that bind said VEGFΔ5 polypeptide at the surface of said cells and/or that modulate the activity of the VEGFΔ5 polypeptide. Such a method may comprise contacting a test compound with a recombinant host cell comprising a reporter construct, said reporter construct comprising a reporter gene whose splicing is under the control of nucleic acid sequences implied in the skipping of VEGF exon5, and selecting the test compounds that modulate (e.g. stimulate or reduce) splicing of the reporter gene.

A polypeptide of the invention can be used to screen libraries of compounds in any of a variety of drug screening techniques. Suitable compounds may be isolated from, for example, cells, cell-free preparations, chemical libraries or natural product mixtures.

These agonists or antagonists may be natural or modified substrates, ligands, enzymes, receptors or structural or functional mimetics. For a suitable review of such screening techniques, see Coligan et al., [71].

The above methods may be conducted in vitro, using various devices and conditions, including with immobilized reagents, and may further comprise an additional step of assaying the activity of the selected compounds in a model of a VEGF-related disorder, such as an animal model.

Further preferred assays methods are described in the art.

Definitions

The practice of the present invention will employ conventional techniques of molecular biology, microbiology, recombinant DNA technology and immunology, which are within the skill of those working in the art. Such techniques are explained fully in the literature. Examples of particularly suitable texts for consultation include the following: Sambrook Molecular Cloning; [72]; DNA Cloning, Volumes I and II [73]; Oligonucleotide Synthesis [74]; Nucleic Acid Hybridization [75]; Transcription and Translation [76]; Animal Cell Culture [77]; Immobilized Cells and Enzymes [78];A Practical Guide to Molecular Cloning [79]; the Methods in Enzymology series [80], especially volumes 154 & 155; Gene Transfer Vectors for Mammalian Cells [81]; Immunochemical Methods in Cell and Molecular [82]; Protein Purification: Principles and Practice, [83]; and Handbook of Experimental Immunology, Volumes I-IV [84]. Further examples of standard techniques and procedures which may be employed in order to utilise the invention are given in patent applications such as WO2005/085285, WO2004/043389 and WO03/018621, the contents of which are incorporated herein in their entirety. It will be understood that this invention is not limited to the particular methodology, protocols, cell lines, vectors and reagents described.

Various aspects and embodiments of the present invention will now be described in more detail by way of example, with particular reference to the VEGFΔ5 polypeptides. It will be appreciated that modification of detail may be made without departing from the scope of the invention.

### BRIEF DESCRIPTION OF DRAWINGS

FIG. 1: Schematic representation of various VEGF isoforms and their constituent exons.

FIG. 2: Genotoxic agents induce the expression of VEGF111. The indicated cells were treated with UV-B or camptothecin (30 mJ or 1 μM, respectively, except otherwise indicated) for 24 hours (except otherwise indicated) and VEGF and VEGF111 mRNA were measured by RT-PCR. (A) Polyacrylamide gel electrophoresis of RT-PCR products of VEGF (upper panel), VEGF111 (middle panel) and 28S rRNA (lower panel). Arrows in upper panel show the amplification product of VEGF111. The drawings on the right represent the exons (not to scale) in the corresponding VEGF isoforms. C: control cells; Cp: camptothecin treatment; M: 50 bp molecular weight marqueurs; R: cells rinsed with red phenol-free medium; UV: UV-B irradiation; -: non template control; arrowheads: RT-PCR products of synthetic RNA added to the test tubes to monitor reaction efficiency [86,87]. (B to E) Dose-response analysis (B and D) and kinetic of induction (C and E) of VEGF111 mRNA level in MCF7 cells irradiated by UV-B (B and C) or treated with camptothecin (D and E). The expression of VEGF111 is expressed as the percentage of the signal corresponding to its mRNA taking the overall various VEGF isoforms mRNA, as measured in upper panel of (A) as 100%.

FIG. 3: Expression of recombinant VEGF in HEK293 cells. HEK293 cells were transformed with vectors enabling expression of VEGF111, VEGF121 and VEGF165. Conditioned mediums were analysed by western blotting before and after treatment with PNGase to digest sugar moieties.

FIG. 4: VEGF111 is biologically active in vitro. A and B: VEGF111 activates VEGF-R2 (A) and ERK1/2 (B) in HUVEC. HUVEC were treated with HEK293 conditioned mediums containing VEGF111, VEGF121 or VEGF165, commercially available recombinant VEGF165 produced in bacteria (cVEGF) (20 ng/ml each) or fetal calf serum (FCS, 10%) for 5 minutes. Untreated cells (−) or cells treated with conditioned medium of control HEK293 cells (CM) served as control. Total and phosphorylated VEGF-R2 and ERK1/2 were measured by western blotting. C: VEGF111 promotes proliferation of HUVEC. HUVEC were treated with HEK293 conditioned mediums containing VEGF111(black circles), VEGF121 (open triangles) or VEGF165 (black triangles) (10 ng/ml) or conditioned medium from control HEK293 cells (open circles). The DNA was measured in triplicate wells harvested at increasing time of treatment. D: representative microphotograph of -embryoid bodies after immunofluorescent labeling of CD31 as marker of vasculature. Bar: 200 μM. E: A number of microphotographs (of 6 embryoid bodies from 3 independent experiments for each treatment) were taken at random and analyzed by 5 investigators not aware of the treatments, and a score (from 0 to 3, where 0 indicates lack of vascular labeling, and 3 maximum labeling) was given to each sample. F: CD31 mRNA level was measured in embryoid bodies (10 samples in 3 independent experiment for each group) as described in Material and Methods. Statistical analysis was performed using the homoscedastic Student T test. *: p<0.05; **: p<0.01; ***: p<0.001

FIG. 5: VEGF is biologically active in vivo. Mice were injected with Matrigel containing HEK293 cells expressing reVEGF111 or 165, or transfected with the empty vector, and sacrificed after 3 weeks. A: Photographs of one representative mouse (out of six) before dissection. The dotted lines delimit the surface of high vascularization. B: Photographs of one representative mouse (out of six) after dissection. Upper panel: the white dotted lines delimit the matrigel plugs, and the arrows indicate the lateral thoracic vein. Bottom panel: detail of the matrigel plugs and surrounding tissues. Black arrows indicate highly vascularized tissues surrounding the VEGF111 expressing matrigel plug. C: Number of vessels at the periphery of the tumors. The vessels were counted on a number of paraffin sections of control plugs (n=20), plugs 111 (n=6) and 165 (n=6). D: Expression of CD31 mRNA in the tumors: the mRNA were measured by RT-PCR in the control plugs (n=21) and plugs 111 (n=6) and 165 (n=6). The data were corrected by the signals obtained for the 28S rRNA. Cont: control plugs; **: p<0.0001 (versus control plugs). Statistical analysis was performed using the homoscedastic Student T test.

FIG. 6: VEGF111 is resistant to plasmin degradation. Recombinant VEGF165, VEGF121 or VEGF111 were produced in HEK293 cells. A: Conditioned media containing 20 ng of VEGF were treated with plasmin (0.04 U, 0.08 U, 0.16 U or 0.32 U/ml) at 37° C. for 4 hours and analysed by western blotting using anti-VEGF antibodies. The figure depicts the percentage of degradation of each VEGF by comparison with untreated samples. B: HUVEC were treated with recombinant VEGF111, VEGF121 or VEGF165 produced in HEK293 cells, or with commercially available VEGF165 (cVEGF) or the carrier alone, untreated or treated with plasmin (0.32 units at 37° C. for 4 hours), and cell multiplication was measured by incorporation of ³H-thymidine.

FIG. 7: VEGF111 is resistant to degradation by fluids collected from chronic wound. A: Conditioned media containing 20 ng of recombinant VEGF as produced above were treated with chronic wound fluid collected in one patient (20 μl at 37° C.) for indicated times and analysed by western blotting using anti-VEGF antibodies. B: HUVEC were treated with recombinant VEGF111, VEGF121 or VEGF165 produced in HEK293 cells, or with commercially available VEGF165 or the carrier alone, untreated or treated with fluids from chronic wounds (20 μl at 37° C. for 24 hours), and cell multiplication was measured by incorporation of ³H-thymidine.

FIG. 8: VEGF111 is expressed in blood cells treated with camptothecin (1 μM) ex vivo. Double arrowhead: internal standard. Vertical arrow indicates amplification products of VEGF111.

FIG. 9: Depicts the level of VEGF111 mRNA expressed in percentage versus the control condition taken as 100%. 1: control condition; 2: cells treated with the carrier alone; 3: cells transfected with irrelevant siRNA; 4: cells transfected with siRNA VEGF111(A); 5: cells transfected with siRNA VEGF(B); 6: cells transfected with siRNA(C) (see Table II).

FIG. 10: Depicts the percentage of inhibition of VEGF111 mRNA expression in MCF7 cells treated with UV-B or camptothecin. Caffeine is an inhibitor of ATM/ATR, PD98059 of MEK, SB203580 of p38, SP600125 of JNK, calyculin of protein phosphatases 1 and 2A, and sc-514 of IKK-β.

FIG. 11: Coding sequence of VEGF-A cDNA, with exon boundaries. The figure depicts the coding sequence of VEGF206 isoform cDNA, encoded by the 8 exons.

FIG. 12: Coding sequence of VEGF111 cDNA, with exon boundaries.

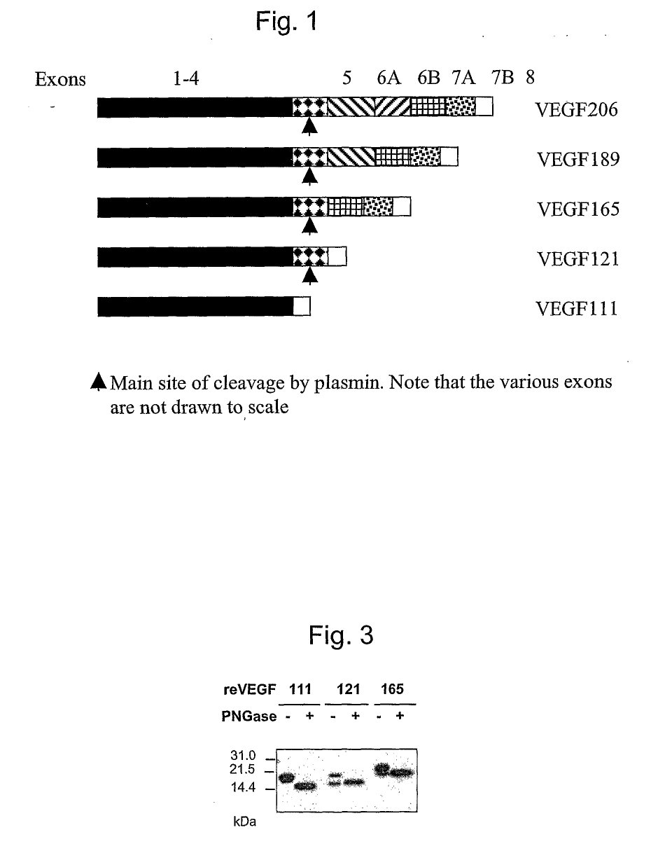

TABLE 1

| Prim- | | |
|---|---|---|
| er | mRNA | Primers |
| P1 | VEGF | CCTGGTGGACATCTTCCAGGAGTA |
| (Fwd) | | |
| P2 | | CTCACCGCCTCGGCTTGTCACA |
| (Rev) | | |
| P3 | VEGF | CACACGCGGCCGCCGAAACCATGAACTTTCTGCTGTC |
| (Fwd) | | |
| P4 | (full- | ACACAGCTAGCTCACCGCCTCGGCTTGTCACA |
| (Rev) | length) | |
| P1 | VEGF111 | CCTGGTGGACATCTTCCAGGAGTA |
| (Fwd) | | |
| P5 | (Speci- | CTCGGCTTGTCACATCTGCATTCA |
| (Rev) | fic) | |
| P6 | 28S | GTTCACCCACTAATAGGGAACGTG |
| (Fwd) | rRNA | |
| P7 | | GATTCTGACTTAGAGGCGTTCAGT |
| (Rev) | | |
| P8 | CD31 | CAAGGCGATTGTAGCCACCTCCA |
| (Fwd) | | |
| P9 | | CCAACAACTCCCCTTGGTCCAGA |
| (Rev) | | |

### MODES FOR CARRYING OUT THE INVENTION

### Example 1

### Materials and Methods

Cell Culture

Normal human primary skin fibroblasts were obtained by explantation from a young healthy donor. MCF7, HaCat, MDA-MB231, were cultured in DMEM containing FCS (10%, Cambrex, Petit-Rechain, Belgium), glutamine (2 mM), ascorbic acid (50 μg/ml), penicillin and streptomycin; Human umbilical vein endothelial cells (HUVEC) were cultured in MCDB-131 medium (InVitrogen) complemented with 20% of FCS, 2 mM glutamine, 5.8 U/ml of heparin (Sigma), penicillin and streptomycin on a coat of 0.2% gelatin. All the cultures were kept at 37° C. under 5% CO2.

Antibodies and Western Blots

The following antibodies were used: anti-ERK1/2 (rabbit polyclonal) and anti phospho-ERK1/2 (mouse monoclonal) were from Sigma (St Louis, Mich.); anti-VEGFR2 (rabbit polyclonal), and anti-VEGF (rabbit polyclonal), were from Santa Cruz (Santa Cruz, Calif.); anti-phospho VEGFR2 (rabbit polyclonal) was from Calbiochem (San Diego, Calif.); rat anti-mouse CD31 from BD Bisoscience, and biotin-labelled anti-rat antibodies, secondary antibodies conjugated with horseradish peroxidase and streptavidin-FITC were from Dako (Glostrup, Denmark).

The proteins were migrated onto polyacrylamide gels and transferred onto PVDF transfer membranes (NEN, Boston, Mass.) by electroblotting. The membranes were blocked by non-fat dry milk (3% in PBS-tween buffer) and probed with first and horseradish peroxidase-conjugated secondary antibodies. Signals were detected by chemiluminescence using a ECL Western Blotting Analysis system (Amersham, Little Chalfont, England) and X-ray film exposure and quantified using a Fluor-S MultiImager (BioRad, Hercules, Calif.).

RNA Purification

Total RNA were purified from cell culture using a High Pure RNA Isolation Kit (Roche Diagnostic, Mannheim, Germany), from early mouse embryos (days 6 to 9) using a High Pure RNA Tissue Kit (Roche Diagnostics) after grinding in lysis solution with a Dounce homogeneiser, and from older mouse embryos and adult mouse or human tissues by cesium chloride floatation [85] after crushing at liquid nitrogen temperature.

RT-PCR Amplification

The RT-PCR amplifications were performed in an automated system (GeneAmp PCR System 2400 or 9600, Perkin Elmer, Norwalk, Conn.) using the GeneAmp Thermostable rTth Reverse Transcriptase RNA PCR kit (Perkin Elmer), 10 ng of total RNA and the different pairs of primers (5 pmole each, see Table 1). For amplification of VEGF isoforms mRNA and 28S rRNA, a known copy number of an internal standard RNA were included in each sample to monitor the efficiency of the reaction [86,87]. The RT step (70° C., 15 min.) was followed by a 2 min. incubation at 95° C. for denaturation of RNA/DNA heteroduplexes, PCR amplification and final elongation for 2 min. at 72° C. The conditions for PCR amplification were 94° C. for 15 sec., 66° C. for 20 sec., 72° C. for 10 sec. for 28S rRNA and CD31 mRNA, or 94° C. for 20 sec, 66° C. for 30 sec. and 72° C. for one min for VEGF. RT-PCR products were resolved on 10% polyacrylamide gel and analysed using a Fluor-S MultiImager (BioRad) after staining with gelstar dye (FMC BioProducts, Rockland, Me.). Specific detection of VEGF111 isoform mRNA was performed as follow. mRNA was reverse transcribed using oligodT (Eurogentec, Seraing, Belgium) and Superscript II (Invitrogen) as described by the manufacturer. Reverse transcription (42° C., 50 min.) was followed by denaturation of the enzyme at 70° C. for 15 min. VEGF111 cDNA was amplified by PCR using 40 ng cDNA, the oligos P5 and P6 (Table 1) and Taq polymerase (Takara, Shiga, Japan). The conditions for PCR amplification were 94° C. for 15 sec., 70° C. for 20 sec.,72° C. for 10 sec.

Irradiation with Ultraviolet B Light

The cells were seeded (5×10⁵ cells per dish 60 mm diameter or 1×10⁵ cells per dish 30 mm diameter) for 24 hours. The culture medium was replaced by 500 μL or 200 μL of DMEM without phenol red and the cells were irradiated for 45 seconds with UV light (30 mJ/cm2) using two Philips TL 20W/12 lamps in the hood, the cover of the dish being removed. The proportion of UV-A light was about 10% of the total UV light while no UV-C were detected, as measured by a UVX radiometer (UVP Inc, San Gabriel, Calif.). After irradiation the phenol red-free DMEM was replaced by culture medium.

Characterization of the VEGF111 Splice Variant cDNA

VEGF mRNA from UV-irradiated HaCat cells were RT-PCR amplified using P1 and P2 primers. VEGF111 specific product was gel purified and sequenced using a Thermo-sequenase radiolabeled terminator cycle sequence kit (Amersham Biosciences Inc.). Full-length VEGF111 cDNA was obtained by RT-PCR amplification using primers P3 and P4 and sequenced at the core facility of the University.

Recombinant VEGF

RNA purified from UV-irradiated HaCat cells was reverse-transcribed using SuperScriptII and an oligodT primer. The complete coding sequences of VEGF111, 121 and 165 were amplified with Pwo DNA polymerase (Roche) using P3 (containing a NotI restriction site at its 5′ end) as forward primer and P4 (containing a NheI restriction site at its 5′ end) as reverse primer. After NotI/NheI digestion and gel purification, the PCR products were ligated (Ligation Kit version II, TaKaRa) between the NotI and NheI sites of a pCEP4 vector (InVitrogen) containing a modified multiple cloning site. Plasmids were amplified in bacteria and prepared using Plasmid Miniprep Kit (BioRad). HEK293 cells were transfected by 1-2 μg plasmid using FuGene 6 (Roche Molecular Biomedicals) and transformed cells were selected by hygromycin (100 mg/L) for 2-3 weeks. For VEGF production the transformed cells were grown to confluence and the medium was replaced by serum- and hygromycin-free DMEM. After 48 hours, conditioned mediums were centrifuged to remove cell debris.

Cell Multiplication

15000 HUVEC were seeded in gelatin-coated wells of 24 multiwells dishes in the presence of FCS. After three hours, the medium was replaced by fresh medium containing or not VEGF and renewed every two days. Cells were collected at various times and the DNA was measured by fluorimetry on a SpectraMax Gemini XS apparatus (Molecular Devices, England) after labeling with bis-benzimide. In some experiments, ³H-thymidine (1 μM, 2.5 Ci/mol, NEN, Wellesley, Mass., USA) was added to cell cultures at day two and the trichloracetic acid-precipitable radioactivity was measured after 18 hours.

Intracellular Calcium Measurement

HUVEC were plated for 18 hours in MCDB-131 medium containing 20% FCS in borosilicate culture chambers (Lab-Tek®, Nunc, Rochester N.Y.) coated with gelatin, rinsed with serum-free medium, incubated with the fluorophore Fluo3-AM (10 μM, Molecular Probes, Eugene, Oreg.) in serum-free medium for two hours and washed. The observation of the fluorescence emitted by the Fluo3-labeled cells started 5 minutes after the last washing. Microscope fields randomly selected were examined by a confocal microscope (Meridian, Akemos, Mich.). The Fluo3-loaded cells were excited by an Argon LASER at 488 nm and the emitted fluorescence recorded at 530 nm in each cell of the field by real time imaging. Image processing and data computing were performed using the Meridian software. The intensity of the emitted fluorescence was recorded every second during 4 minutes. As the overall fluorescence intensity varied from cell to cell, the level of fluorescence of each cell at the beginning of the recording was normalised to one arbitrary unit. The baseline of resting cells spontaneously oscillated by ±10% around the level of the first image acquisition. After a 20 second period of baseline recording, 10-20 μL of conditioned medium from HEK293 cells expressing or not VEGF111, VEGF121 or VEGF165 (10 ng) were added gently on the cells under microscopic examination. A 20% rise above the baseline was considered as a significant calcium rise and the cell regarded as responsive. The results were expressed as the percentage of responding cells.

Deglycosylation of VEGF

Conditioned medium of HEK293 cells expressing VEGF165, VEGF121 or VEGF111 or of control cells were treated with N-glycosidase F (PNGase F, New England BioLabs, Ipswich, Mass.) as described by the manufacturer. The electrophoretic pattern of the various VEGF isoforms before and after treatment was determined by western blotting.

In Vitro Angiogenesis

Embryoid bodies were formed as previously described [⁸⁸]. Briefly, undifferentiated ES CGR8 cells were aggregated for 4 days in a 20 μl drop of DMEM supplemented with 10% FCS, 0.1 mM non-essential amino-acids and 0.1 mM β-mercaptoethanol, and further kept in culture on gelatin-coated coverslips for 6 days in the same medium containing the various recombinant VEGF isoforms (25 ng/ml). For immunohistochemistry the embryoid bodies were fixed in methanol and incubated with rat anti-mouse CD31 antibodies, biotin-conjugated anti-rat IgG and streptavidin/FITC. Immunostaining was observed by confocal microscopy (Leica, Wetzlar, Germany).

In Vivo Angiogenesis

Control HEK293 cells (transfected with the empty vector, 2×10⁶) or expressing human recombinant VEGF111, 121 or 165 were mixed with 200 μl matrigel depleted in growth factors (Becton Dickinson, adresse) and sub-cutaneously injected in the flanks of nude mice (6 weeks old Swiss Nu/Nu). After 3 weeks the mice were sacrificed according to the ethical policy of our Institute. The matrigel plugs and surrounding tissues were inspected, and the matrigel plugs were dissected. A part was fixed in formaldehyde and mounted in paraffin and sections were analyzed after staining with hematoxillin and eosin. Another part was used for preparation of total RNA.

### Example 2

### Genotoxic Agents Induce Expression of a New VEGF Isoform Lacking Exon 5

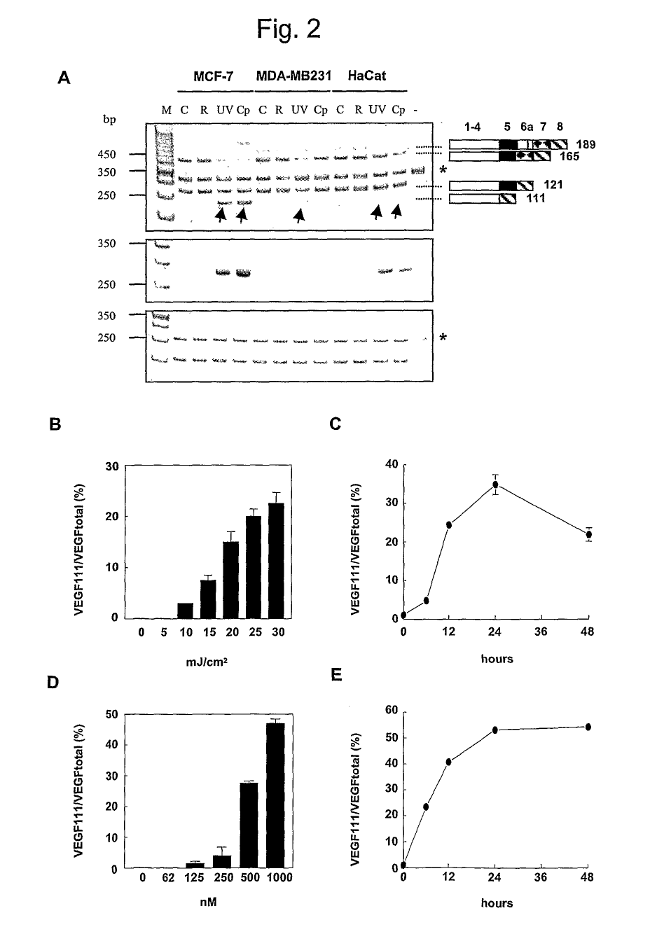

The effect of irradiation by UV-B (30 mJ/cm2) on the expression of VEGF mRNA isoforms by HaCat cells (immortalized human keratinocytes), MDA-MB-231 cells and MCF-7 cells (two transformed human breast epithelial cell lines). The mRNA encoding the various VEGF isoforms was measured by RT-PCR. Primers (P1 and P2, see Table 1) were chosen on exons 3 and 8 of the VEGF gene to enable the amplification and size-based discrimination of the various isoforms of human and mouse VEGF known at the time of the experiment. They also allow amplification of VEGF mRNA from hamster in spite of one mismatch between the sequence complementary to the reverse primer in human and mouse mRNA and in hamster mRNA. VEGF189, VEGF165 and VEGF121 mRNA were detected in the control and irradiated cells (FIG. 2A, upper panel). Their level was essentially unaffected or lowered by UV-B irradiation in the three cell lines. Amplification of the 28S rRNA (Primers P6 and P7, see Table 1) was used to control that similar amounts of RNA were amplified (FIG. 2A, lower panel). A fast-migrating RT-PCR product (arrow in FIG. 2A, higher panel) was observed in irradiated MCF7 cells, and at a lower level in irradiated HaCat and MDA-MB-231 cells, but not in control cells. The fast-migrating cDNA amplified from UV-B irradiated HaCat cells was extracted from acrylamide gels, amplified by PCR and sequenced using the primers P1 and P2 (Table 1). Analysis revealed that it contained the expected sequence of exons 3, 4 and 8 of VEGF but missed the sequence encoded by exons 5 to 7. Full-length VEGF cDNA was generated using RNA from UV-treated HaCat cells and the primers P3 and P4 (Table 1). After migration of the reaction products on acrylamide gel the faster-migrating band was excised. Sequencing showed that it has the sequence of VEGF exons 1 to 4 and exon 8 but not of exons 5 to 7 (see FIG. 2A, upper panel). According to the current nomenclature it was named VEGF111 as the sequence encodes a 111 amino-acids long VEGF molecule after excision of the signal peptide. Of note the excision of the 30 base pairs encoded by the exon 5 does not change the reading frame of the downstream sequence.

As the junction between exons 4 and 8 is specific to VEGF111 mRNA we amplified this isoform by using a reverse primer (P5) sitting astride these exons, the forward primer (P1) sitting on exon 3. A product of the expected size was detected after a two step RT-PCR amplification using total RNA from UV-B irradiated cells, but not from control cells, reflecting the amplification of the fast-migrating product observed above (FIG. 2A middle panel).

A dose-response analysis of the expression of VEGF111 mRNA upon treatment with UV-B up to 30 mJ was performed in MCF7 cells. The expression of VEGF111 progressively increased with the energy of irradiation up to a maximum of 25% of the overall VEGF mRNA (FIG. 2B). An energy of 30 mJ was therefore chosen in all subsequent experiments. The expression of VEGF111 mRNA was measured in MCF7 cells harvested at various times after irradiation. It was readily detected after 12 hours, peaked at 24 hours and decreased thereafter (FIG. 2C).

Since UV-B has genotoxic effects, the induction of VEGF111 expression by genotoxic pharmacological agents, namely camptothecin, mimosin and mitomycin C was investigated. Camptothecin (1 μM, Sigma), a topoisomerase I poison, induced the expression of VEGF111 in HaCat cells and in MCF7 cells by 24 hours, to a level similar to or higher than that observed after UV-irradiation (FIG. 2A). A dose-response analysis indicated that VEGF111 induction progressively increased with the concentration of camptothecin in MCF7 cells (FIG. 2D). Time-course experiments showed that VEGF111 induction was already detected after 6 hours and reached a plateau after 24 hours (FIG. 2E). L-mimosin (5 mM) and mitomycin C (100 mg/ml) also induced the expression of VEGF111 mRNA at a level similar to that observed in UV-B treated MCF-7 cells.

### Example 3

### Recombinant VEGF111—Glycosylation and Proteolytic Degradation Resistance

Glycosylation

The cDNA of VEGF111, VEGF121 and VEGF165 were cloned by RT-PCR and introduced downstream of a cytomegalovirus promoter in plasmid pCEP4. The resulting vectors were transfected in HEK293 cells and the transformed cells were selected by treatment with hygromycin. These cells were chosen because of their low intrinsic expression of VEGF and high potency to express recombinant proteins and transfection efficiency. Recombinant VEGF165, VEGF121 and VEGF111 (thereafter called reVEGFs for recombinant VEGF produced in eukaryotic cells) were produced in serum-free conditioned medium of HEK293 cells and their concentration was measured by ELISA. Conditioned media containing 20 ng VEGF were analysed by western blot in reducing conditions prior or after treatment with PNGase to cleave sugar moieties (FIG. 3). In the absence of treatment a signal appeared as a single band (mw ˜18 kDa) on the autoradiogram in the media of cells expressing reVEGF111, and as two bands (mw ˜17 kDa and 21 kDa, and 22 kDa and 26 kDa) in the media of cells expressing reVEGF121 and reVEGF165, respectively. No signal was detected in conditioned medium from control cells. As VEGF contains one glycosylation site the bands of lower mobility corresponded to glycosylated form of the reVEGF121 and reVEGF165. Only the bands of higher mobility were observed after deglycosylation of the samples with PNGase, and these bands have the approximate sizes expected on the basis of their amino-acid sequences. Moreover reVEGF165 produced in HEK293 cells has identical mobility as rbVEGF165 (for recombinant VEGF produced in bacteria). Deglycosylation of reVEGF111 produced a band of higher mobility (˜14 kDa), suggesting that the reVEGF111 is completely glycosylated in HEK293 cells. Again this band had the approximate size expected on the basis of VEGF111 amino-acid sequence.

Plasmin Resistance

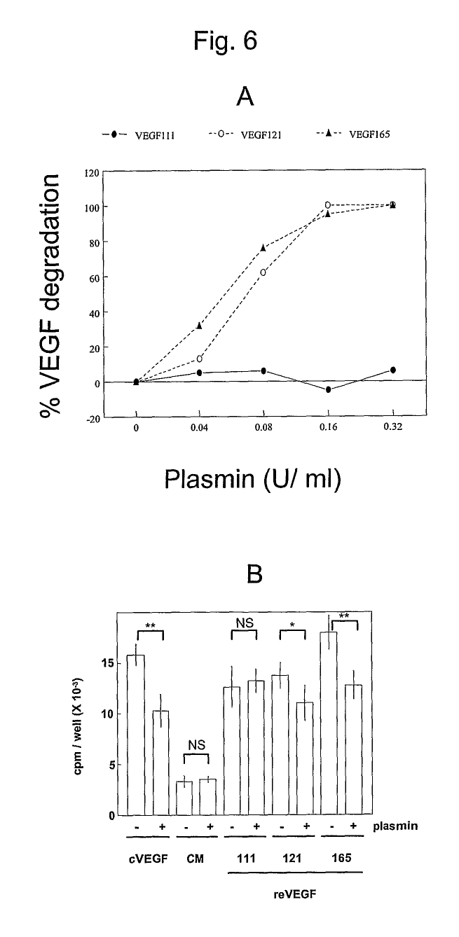

VEGF is susceptible to degradation by plasmin (Keyt et al., 1996), the main site of cleavage being identified as Arg110-Ala111, encoded by exon 5, suggesting that VEGF111 might be resistant to plasmin. reVEGFs (20 ng each) were treated with plasmin (0.04 to 0.32 units/ml) for 4 hours. To facilitate the analysis of the electrophoretic patterns they were further treated with PNGase as described above and the products of the reactions were analysed by western blotting. Data revealed that reVEGF121 and reVEGF165 were progressively degraded, while reVEGF111 treated in parallel was unaffected. FIG. 6A depicts the percentage of degradation of each VEGF by comparison with untreated samples. The dose of plasmin that cleaves VEGF by 50% in the conditions of the present assay is comprised between 0.05 and 0.07 U/ml for VEGF165 and VEGF121, and above 0.32 U/ml for VEGF111. FIG. 6B depicts the effect of plasmin treatment on the mitogenic activity of the recombinants VEGF on HUVEC.

Resistance to Degradation by Chronic Wound Fluids

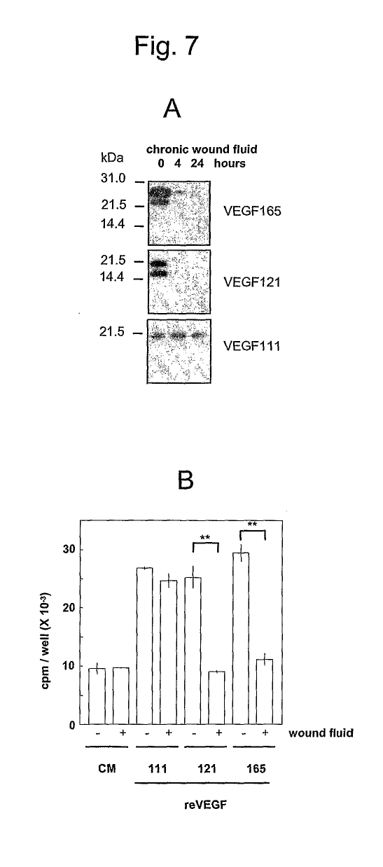

Conditioned media containing 20 ng of recombinant VEGF as produced above were treated with chronic wound fluid collected in one patient (20 μl at 37° C.) for indicated times and analysed by western blotting using anti-VEGF antibodies (FIG. 7A). VEGF111 showed no detectable degradation after 24 hrs. FIG. 7B depicts the effect of treatment with chronic wound fluids on the mitogenic activity of the recombinants VEGF on HUVEC.

### Example 4

### rVEGF111 is Biologically Active In Vitro and In Vivo

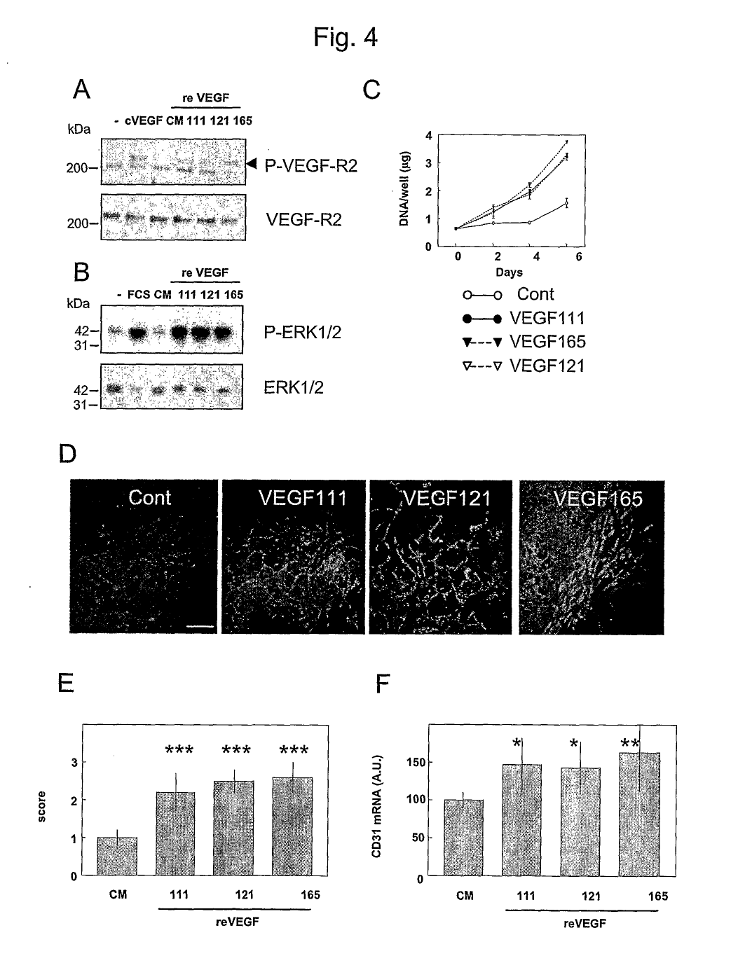

VEGF induces a number of effects in endothelial cells, including autophosphorylation of its receptors [18], activation of the ERK1/2 MAPkinases pathways, induction of calcium transients and increased proliferation. The in vitro effects of recombinants VEGFs on HUVEC were compared. HUVEC were starved overnight and treated for five min. with reVEGFs. By immunoblotting, we observed that the reVEGF165 and rbVEGF165 were able to phoshorylate the VEGF-R2 in HUVEC as expected. Similarly reVEGF111 and reVEGF121 phosphorylated the VEGF-R2 in HUVEC, although at a lower level than VEGF165 (FIG. 4A). Conditioned medium from HEK293 cells transfected with the empty vector had no effect. To further confirm the biological activity of VEGF111 the activation of ERK1/2 signalling pathways was investigated by western blotting using monoclonal phospho-specific antibodies. HUVEC were starved overnight and treated with the three reVEGF isoforms, as well as rbVEGF165, for 5 min. In all cases the phosphorylation of ERK1/2 was induced in HUVEC (FIG. 4B). Probing of the membranes with rabbit polyclonal anti-ERK1/2 antibodies demonstrated that similar amounts of proteins were present in the control and VEGF-treated samples. Against, control conditioned medium had no effect on the ERK1/2 phosphorylation status in these cell lines.

VEGF was shown to induce transient increases of intracellular free calcium concentration in endothelial cells [20]. The induction of calcium transients by the various reVEGFs was investigated on HUVEC by real-time fluorescent microscopy as described [89]. As the calcium concentration in resting cells spontaneously varied by about 10% around the baseline, cells were defined as responsive when the treatments induced a peak of calcium at least 20% above the baseline. HUVEC were treated with conditioned mediums from control HEK293 cells or cells expressing VEGF111, VEGF121 and VEGF165. Control conditioned medium induced calcium transients in about 15% of HUVEC, while the mediums containing each of the three reVEGFs induced transients in 70% of the cells, against demonstrating their biological activity.

The effect of reVEGFs on the proliferation rate of HUVEC was tested. HUVEC were treated with conditioned medium control or containing the reVEGF, and the amount of DNA was measured after increasing time of culture, the mediums being renewed every two days. reVEGF165 induced a 2.5 fold increase of proliferation rate as compared to control conditioned medium (FIG. 4C). VEGF111 and VEGF121 also stimulated proliferation, although by a slightly lower factor (2 fold).

The possible induction of angiogenesis by VEGF111 was tested in an embryoid body model. Mouse ES cells were cultured in the absence or in the presence of the three reVEGF. Embryoid bodies (6 from 3 independent experiments) were fixed at day 6, and labeled by anti—1.6 fold) the mRNA of CD31 in comparison to control medium (FIG. 4F).

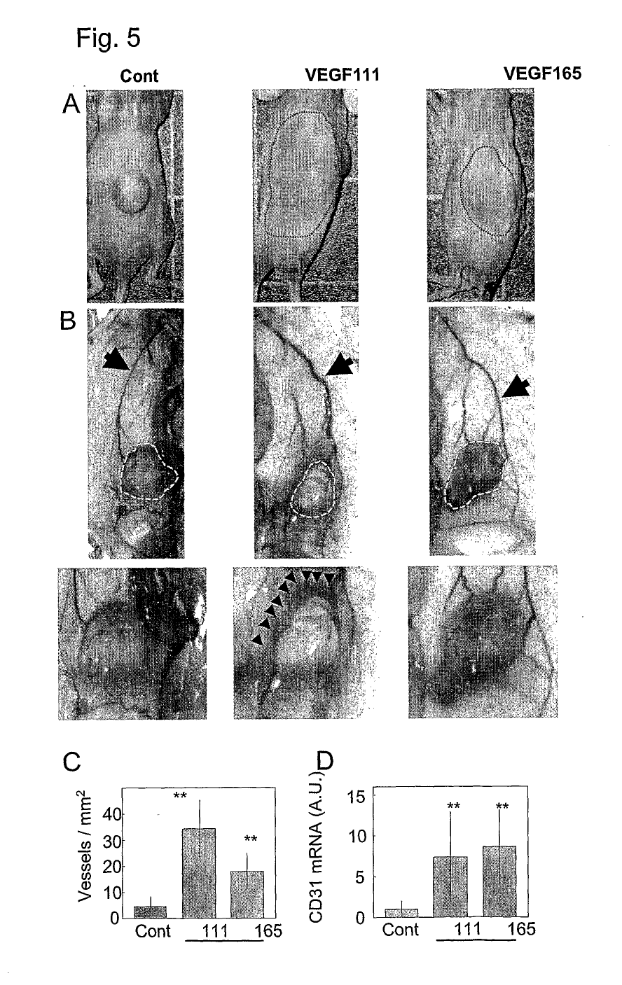

Nude mice (6 per group) were injected with matrigel containing control HEK293 in one flank (control plugs, Cont) and HEK293 expressing VEGF111 (plugs 111) or VEGF165 (plugs165) in the other flank. The levels of human VEGF were measured in the blood collected in the heart after sacrifice. Human VEGF was absent in mice injected with control cells in both flanks, but was detected in all the mice injected with cells expressing human VEGF111 or VEGF165. These data were confirmed by the measurements of the VEGF mRNA in the matrigel plugs. Tumors were induced at the site of injection in nearly all mice. However they were more diffuse on plugs 111 and 165 as compared to control plugs. Vascularization in or under the skin was observed at the site of injection (delineated by dotted black line in FIG. 5A): it was induced with plugs 111 and 165, but never on control plugs. After dissection we observed that the control plugs and surrounding tissues were not or poorly vascularized. Vascularization was significantly induced around the plugs 111 (6/6 mice), while the plugs themselves were poorly vascularized (FIG. 5B). By contrast the plugs 165 (6/6 mice) where highly vascularized, but not or poorly in its vicinity. In most cases the lateral thoracic vein and afferent vessels were enlarged in the side injected with VEGF expressing cells as compared to control cells.

The plugs and the adherent skin were dissected and the number of vessels in the plugs and between the plug and the skin were counted. It was low in all plugs, whatever the cells that they contained, and at the periphery of control plugs (FIG. 5C), but increased at the periphery of plugs 111 and 165. Significantly (p<0.02), the number of vessels was higher at the periphery of plugs 111 than plugs 165. Total RNA was extracted from the matrigel plugs. The VEGF mRNA was undetectable in control plugs while plugs 111 and 165 express high levels of the mRNA of the corresponding VEGF. The CD31 mRNA (FIG. 5D) and von Willebrand factor mRNA were increased in plugs 111 and 165 as compared to control plugs.

### Example 5

### Expression of VEGF111 in Human and Mouse Tissues

The expression of VEGF111 was measured in a number of normal adult tissues from human (prostate, breast, brain, lung, cervix, kidney, endometrium and skin) and mice (same organs as in human plus heart, liver, bone, spleen, eye, stomach, muscle, intestine, tendon and placenta), as well as 6 to 18 day old total mice embryos. VEGF111 was never detected in these samples whereas VEGF121, VEGF165 and VEGF189 isoforms were easily detected, though at variable levels.

### Example 6

### Inhibition of UV-B and Camptothecin-Induced VEGF111 Expression by siRNA

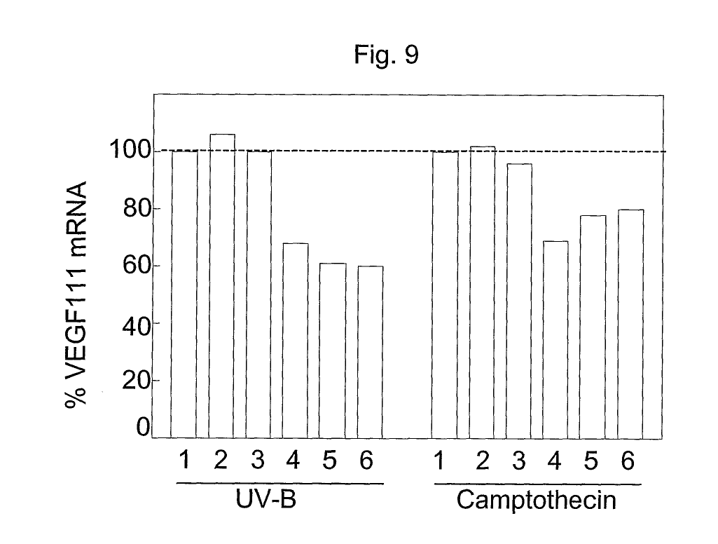

MCF7 cells were transfected by siRNA (20 nM) using a calcium phosphate procedure [21]. After 48 hours they were irradiated by UV-B (30 mJ/cm2) or treated with camptothecin (1 μM) and harvested 24 hours later. VEGF mRNA were measured by RT-PCR. FIG. 9 depicts the level of VEGF111 mRNA expressed in percentage versus the control condition taken as 100%. Table 2 shows the sequence of the siRNA used.

TABLE 2

| siRNA | Sequence |
|---|---|
| VEGF111(A) | GUGAAUGCAGAUGUGACAAdTdT |
| | dTdTCACUUACGUCUACACUGUU |
| VEGF111(B) | UGUGAAUGCAGAUGUGACAdTdT |
| | dTdTACACUUACGUCUACACUGU |
| VEGF111(C) | AUGUGAAUGCAGAUGUGACdTdT |
| | dTdTUACACUUACGUCUACACUG |

### Example 7

### Inhibition of VEGF111 Expression by Pharmacological Agents

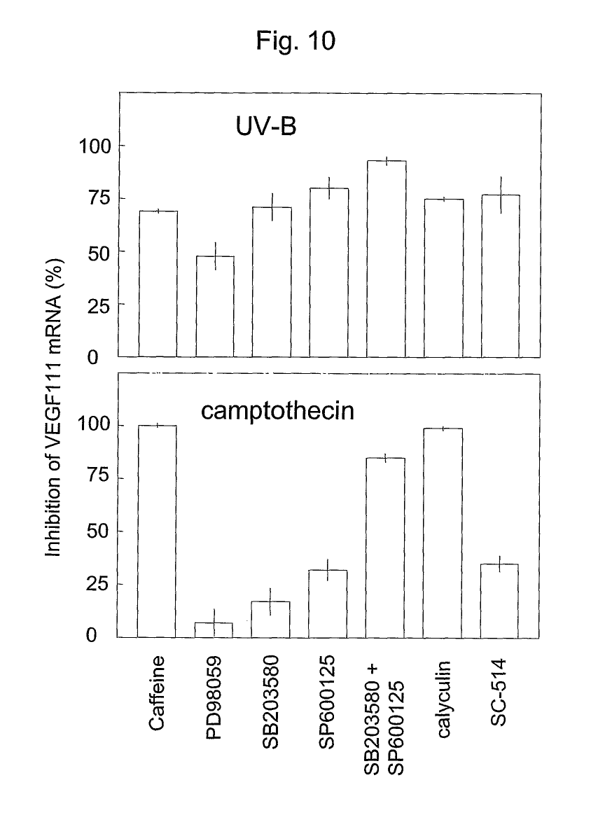

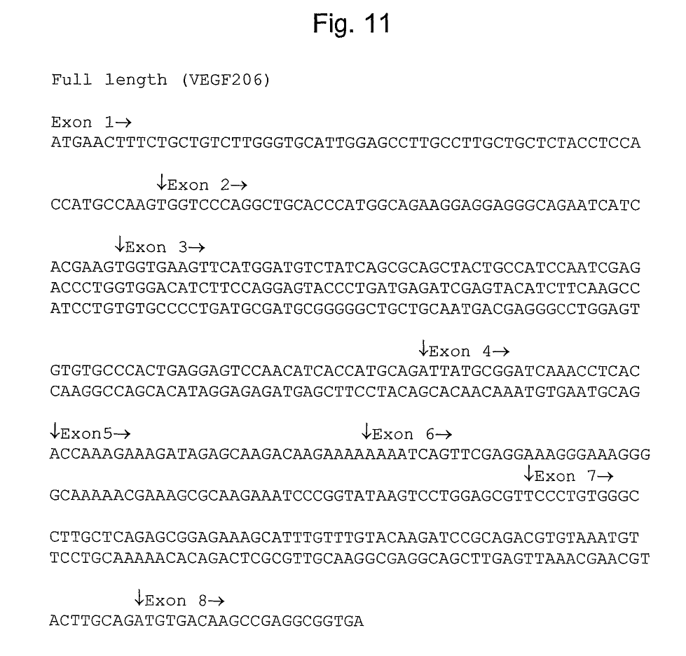

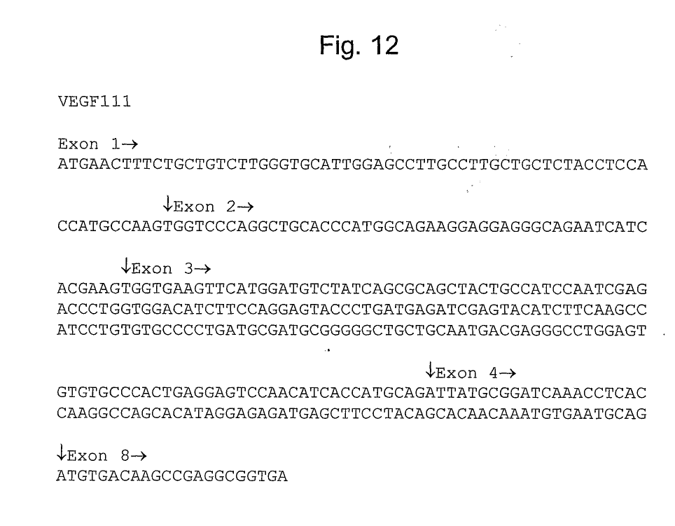

MCF7 cells were treated by inhibitors of ATM/ATR (caffeine), IKK-beta (sc-514), p38 (SB203580), MEK1 (PD98059), JNK (SP600125) or protein phosphatases 1 and 2A (calyculin), and irradiated by UV-B (30 mJ/cm2) or treated with camptothecin (1 μM) and harvested 24 hours later. VEGF mRNA was measured by RT-PCR. FIG. 10 depicts the percentage of inhibition of VEGF111 mRNA expression as compared to cells treated with UV-B or camptothecin alone.

### Example 8

### VEGF111 is Expressed in Blood Cells Treated with Camptothecin Ex Vivo

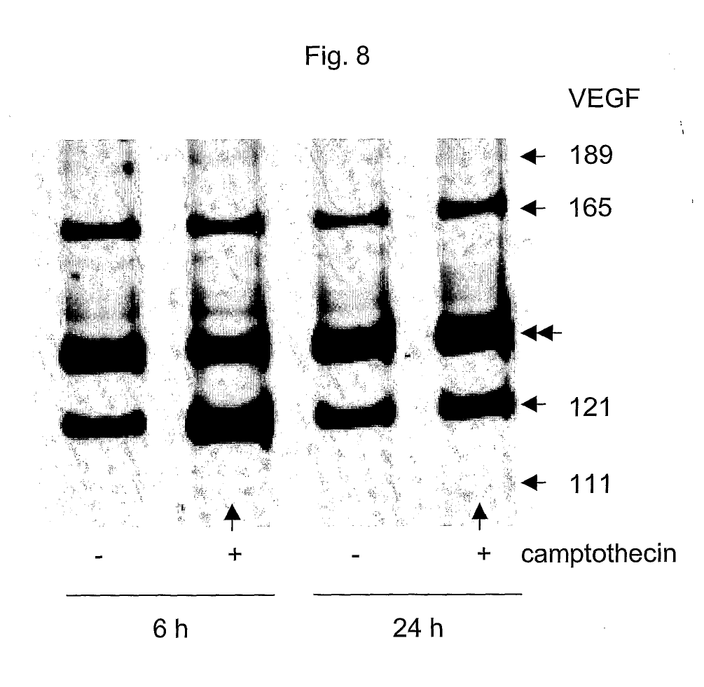

Blood was collected from a healthy donor, and cells were collected by centrifugation. Red blood cells were lyzed and the remaining cells were treated or not with camptothecin (1 μM) for 6 and 24 hours. Untreated cells were also investigated directly after collection. Total VEGF mRNA was measured by RT-PCR as described. Data indicated that VEGF111 mRNA is expressed in camptothecin treated cells (FIG. 8).

### REFERENCES

[1] Dvorak H F (2000) VPF/VEGF and the angiogenic response. Semin Perinatol. 24:75-8.
[2] Carmeliet P, Ferreira V, Breier G, Pollefeyt S, Kieckens L, Gertsenstein M, Fahrig M, Vandenhoeck A, Harpal K, Eberhardt C, Declercq C, Pawling J, Moons L, Collen D, Risau W, Nagy A (1996) Abnormal blood vessel development and lethality in embryos lacking a single VEGF allele. Nature. 380:435-9.
[3] Ferrara N, Carver-Moore K, Chen H, Dowd M, Lu L, O'Shea K S, Powell-Braxton L, Hillan K J, Moore M W (1996) Heterozygous embryonic lethality induced by targeted inactivation of the VEGF gene. Nature 380:439-42.
[4] Soker S, Takashima S, Miao H Q, Neufeld G, Klagsbrun M (1998) Neuropilin-1 is expressed by endothelial and tumor cells as an isoform-specific receptor for vascular endothelial growth factor. Cell. 92:735-45.
[5] Bando H, Weich H A, Brokelmann M, Horiguchi S, Funata N, Ogawa T, Toi M (2005) Association between intratumoral free and total VEGF, soluble VEGFR-1, VEGFR-2 and prognosis in breast cancer. Br. J. Cancer 92:553-561
[6] Leung D W, Cachianes G, Kuang W J, Goeddel D V, Ferrara N. (1989) Vascular endothelial growth factor is a secreted angiogenic mitogen. Science. 1989 Dec. 8; 246(4935):1306-9.
[7] Houck K A, Ferrara N, Winer J, Cachianes G, Li B, Leung D W (1991) The vascular endothelial growth factor family: identification of a fourth molecular species and characterization of alternative splicing of RNA. Mol Endocrinol. 5:1806-14
[8] Poltorak Z, Cohen T, Sivan R, Kandelis Y, Spira G, Vlodaysky I, Keshet E, Neufeld G. (1997) VEGF145, a secreted vascular endothelial growth factor isoform that binds to extracellular matrix. J Biol Chem. 272:7151-8.
[9] Jingjing L, Xue Y, Agarwal N, Roque R S (1999) Human Muller cells express VEGF183, a novel spliced variant of vascular endothelial growth factor. Invest Ophthalmol Vis Sci. 40:752-9.
[10] Whittle C, Gillespie K, Harrison R, Mathieson P W, Harper S (1999) Heterogeneous vascular endothelial growth factor (VEGF) isoform mRNA and receptor mRNA expression in human glomeruli, and the identification of VEGF148 mRNA, a novel truncated splice variant. Clin Sci (Lond). 97:303-12.
[11] Lange T, Guttmann-Raviv N, Baruch L, Machluf M, Neufeld G. (2003) VEGF162, a new heparin-binding vascular endothelial growth factor splice form that is expressed in transformed human cells. J Biol Chem. 278:17164-9.
[12] Woolard J, Wang W Y, Bevan H S, Qiu Y, Morbidelli L, Pritchard-Jones R O, Cui T G, Sugiono M, Waine E, Perrin R, Foster R, Digby-Bell J, Shields J D, Whittles C E, Mushens R E, Gillatt D A, Ziche M, Harper S J, Bates D O (2004) VEGF165b, an inhibitory vascular endothelial growth factor splice variant: mechanism of action, in vivo effect on angiogenesis and endogenous protein expression. Cancer Res. 64:7822-35.
[13] Keyt B A, Berleau L T, Nguyen H V, Chen H, Heinsohn H, Vandlen R, Ferrara N (1996) The carboxyl-terminal domain (111-165) of vascular endothelial growth factor is critical for its mitogenic potency. J Biol Chem. 271:7788-95
[14] Plouet J, Moro F, Bertagnolli S, Coldeboeuf N, Mazarguil H, Clamens S, Bayard F (1997) Extracellular cleavage of the vascular endothelial growth factor 189-amino acid form by urokinase is required for its mitogenic effect. J Biol Chem. 272:13390-6.
[15] Claffey K P, Senger D R, Spiegelman B M (1995) Structural requirements for dimerization, glycosylation, secretion, and biological function of VPF/VEGF. Biochim Biophys Acta. 1246:1-9
[16] Lee S, Jilani S M, Nikolova G V, Carpizo D, Iruela-Arispe M L (2005) Processing of VEGF-A by matrix metalloproteinases regulates bioavailability and vascular patterning in tumors. J Cell Biol. 169:681-91
[17] Lauer G, Sollberg S, Cole M, Krieg T, Eming S A (2002) Generation of a novel proteolysis resistant vascular endothelial growth factor165 variant by a site-directed mutation at the plasmin sensitive cleavage site. FEBS Lett. 531:309-13.
[18] Waltenberger J, Claesson-Welsh L, Siegbahn A, Shibuya M, Heldin C H (1994) Different signal transduction properties of KDR and Flt1, two receptors for vascular endothelial growth factor. J Biol Chem. 269:26988-95
[19] Yu and Sato (1999) MAP kinases, phosphatidylinositol 3-kinase, and p70 S6 kinase mediate the mitogenic response of human endothelial cells to vascular endothelial growth factor. J Cell Physiol. 178:235-46.
[20] Brock T A, Dvorak H F, Senger D R (1991) Tumor-secreted vascular permeability factor increases cytosolic Ca2+ and von Willebrand factor release in human endothelial cells. Am J Pathol. 138:213-21
[21] Deroanne C F, Colige A C, Nusgens B V, Lapiere C M. (1996) Modulation of expression and assembly of vinculin during in vitro fibrillar collagen-induced angiogenesis and its reversal. Exp Cell Res. 224:215-23.
[22] Computational Molecular Biology, Lesk, A. M., ed., Oxford University Press, New York, 1988
[23] Biocomputing. Informatics and Genome Projects, Smith, D. W., ed., Academic Press, New York, 1993
[24] Computer Analysis of Sequence Data, Part 1, Griffin, A. M., and Griffin, H. G., eds., Humana Press, New Jersey, 1994
[25] Sequence Analysis in Molecular Biology, von Heinje, G., Academic Press, 1987
[26] Sequence Analysis Primer, Gribskov, M. and Devereux, J., eds., M Stockton Press, New York, 1991
[27] Gentz et al. 1989, Proc Natl Acad Sci USA, 86:821-4
[28] Wilson et al. 1994, Cell, 37:767-78
[29] Terpe K, 2003, Appl Microbiol Biotechnol, 60:523-33
[30] Usman, N, et al., Curr. Opin. Struct. Biol (1996) 6(4), 527-33
[31] Ferrara N. Nat Rev Cancer. (2002) VEGF and the quest for tumour angiogenesis factors. October; 2(10):795-803.
[32] Herbst R S, Sandler A B (2004) Non-small cell lung cancer and antiangiogenic therapy: what can be expected of bevacizumab? Oncologist 9 Suppl 1:19-26
[33] Rugo H S (2004) Bevacizumab in the treatment of breast cancer: rationale and current data. Oncologist. 9 Suppl 1:43-9
[34] Ferrara N, Hillan K J, Novotny W (2005) Bevacizumab (Avastin), a humanized anti-VEGF monoclonal antibody for cancer therapy. Biochem Biophys Res Commun. 333:328-35.
[35] Chen H X, Gore-Langton R E, Cheson B D (2001) Clinical trials referral resource: Current clinical trials of the anti-VEGF monoclonal antibody bevacizumab. Oncology 15:1017, 1020, 1023-6.
[36] Verheul H M, Pinedo H M (2005) Inhibition of angiogenesis in cancer patients. Expert Opin Emerg Drugs. 10:403-12.
[37] Mulcahy M F, Benson A B 3rd (2005) Bevacizumab in the treatment of colorectal cancer. Expert Opin Biol Ther. 5:997-1005.
[38] Genentech (2005) http://www.fda.gov/medwatch/SAFETY/2005/Avastin_dearhcp.pdf
[39] Kilickap S, Abali H, Celik I ((2003) Bevacizumab, bleeding, thrombosis, and warfarin. J Clin Oncol. 21:3542
[40] Arthritis Res. 2002;4 Suppl 3:S81-90
[41] Nickoloff B J, Mitra R S, Varani J, Dixit V M, Polverini P J. (1994) Aberrant production of interleukin-8 and thrombospondin-1 by psoriatic keratinocytes mediates angiogenesis. Am J Pathol. 1994 April; 144(4):820-8.
[42] Berkner, K. L., Curr. Top. Microbiol. Immunol., 158, 39-66 (1992)
[43] Muzyczka, N., Curr. Top. Microbiol. Immunol., 158, 97-129 (1992)
[44] Singer A. J. and Clark R. A. 1999 N. Engl. J Med. 341:738-746
[45] Alvarez Arroyo M. V., et al. 1998 Kidney Int Suppl. 68:S7-S9.
[46] Jakeman L. B. et al., 1992, J Clin Invest. 89:244-253
[47] Lauer G, Sollberg S, Cole M, Flamme I, Sturzebecher J, Mann K, Krieg T, Eming S A (2000) Expression and proteolysis of vascular endothelial growth factor is increased in chronic wounds. J Invest Dermatol. 115:12-8.
[48] Zhang, Z. G. et al., 2000, J. Clin. Invest, 106:829-838
[49] Krysiak O, Bretschneider A, Zhong E, Webb J, Hopp H, Verlohren S, Fuhr N, Lanowska M, Nonnenmacher A, Vetter R, Jankowski J, Paul M, Schonfelder G (2005) Soluble Vascular Endothelial Growth Factor Receptor-1 (sFLT-1) Mediates Downregulation of FLT-1 and Prevents Activated Neutrophils From Women With Preeclampsia From Additional Migration by VEGF. Circ Res. 2005 Nov. 3
[50] Rogers R. S., et al., Int. J. Impot. Res. 2003, 15:26-37
[51] Elbashir, S M et al., Nature 2001, 411, 494-498
[52] Fernandez & Hoeffler 1998, eds. “Gene expression systems. Using nature for the art of expression”. Academic Press, San Diego, London, Boston, New York, Sydney, Tokyo, Toronto
[53] Davis et al., Basic Methods in Molecular Biology (1986)
[54] “Current Protocols in Molecular Biology”, Ausubel et al. (eds). Greene Publishing Association and John Wiley Interscience, New York, 1989, 1992
[55] Spector, Goldman & Leinwald, 1998
[56] Kohler, G. and Milstein, C., Nature 256: 495-497 (1975)
[57] Kozbor et al., Immunology Today 4: 72 (1983)
[58] Cole et al., 77-96 in Monoclonal Antibodies and Cancer Therapy, Alan R. Liss, Inc. (1985)
[59] Conrad U, Scheller J (2005) Considerations on antibody-phage display methodology. Comb Chem High Throughput Screen. 8:117-26
[60] Jones et al., Nature, 321, 522 (1986)
[61] Verhoeyen et al., Science, 239, 1534 (1988)
[62] Kabat et al., J. Immunol., 147, 1709 (1991)
[63] Queen et al., Proc. Natl. Acad. Sci. USA, 86, 10029 (1989)
[64] Gorman et al., Proc. Natl Acad. Sci. USA, 88, 34181 (1991)
[65] Hodgson et al., Bio/Technology, 9, 421 (1991)
[66] Presta L G, Chen H, O'Connor S J, Chisholm V, Meng Y G, Krummen L, Winkler M, Ferrara N (1997) Humanization of an anti-vascular endothelial growth factor monoclonal antibody for the therapy of solid tumors and other disorders. Cancer Res. 57:4593-9
[67] Saiki et al., Nature, 324, 163-166 (1986)
[68] Bej, et al., Crit. Rev. Biochem. Molec. Biol., 26, 301-334 (1991)
[69] Birkenmeyer et al., J. Virol. Meth., 35, 117-126 (1991)
[70] Van Brunt, J., Bio/Technology, 8, 291-294 (1990)
[71] Coligan et al., Current Protocols in Immunology 1(2):Chapter 5 (1991)
[72] Sambrook Molecular Cloning; A Laboratory Manual, Second Edition (1989)
[73] DNA Cloning, Volumes I and II (D. N Glover ed. 1985)
[74] Oligonucleotide Synthesis (M. J. Gait ed. 1984)
[75] Nucleic Acid Hybridization (B. D. Hames & S. J. Higgins eds. 1984)
[76] Transcription and Translation (B. D. Hames & S. J. Higgins eds. 1984)
[77] Animal Cell Culture (R. I. Freshney ed. 1986)
[78] Immobilized Cells and Enzymes (IRL Press, 1986)
[79] A Practical Guide to Molecular Cloning B. Perbal, (1984)
[80] The Methods in Enzymology series (Academic Press, Inc.),
[81] Gene Transfer Vectors for Mammalian Cells (J. H. Miller and M. P. Calos eds. 1987, Cold Spring Harbor Laboratory);
[82] Immunochemical Methods in Cell and Molecular Biology (Mayer and Walker, eds. 1987, Academic Press, London)
[83] Protein Purification: Principles and Practice, Second Edition, Scopes, (1987) (Springer Verlag, N.Y.)
[84] Handbook of Experimental Immunology, Volumes I-IV (D. M. Weir and C. C. Blackwell eds. 1986).
[85] Chirgwin J M, Przybyla A E, MacDonald R J, Rutter W J (1979) Isolation of biologically active ribonucleic acids from sources enriched in ribonuclease. Biochemistry 18: 5294-5299.
[86] Lambert C A, Colige A C, Munaut C, Lapiere C M, Nusgens B V (2001) Distinct pathways in the over-expression of matrix metalloproteinases in human fibroblasts by relaxation of mechanical tension. Matrix Biol. 20:397-408
[87] Hajitou A, Sounni N E, Devy L, Grignet-Debrus C, Lewalle J M, Li H, Deroanne C F, Lu H, Colige A, Nusgens B V, Frankenne F, Maron A, Yeh P, Perricaudet M, Chang Y, Soria C, Calberg-Bacq C M, Foidart J M, Noel A (2001) Down-regulation of vascular endothelial growth factor by tissue inhibitor of metalloproteinase-2: effect on in vivo mammary tumor growth and angiogenesis. Cancer Res. 61:3450-7
[88] Deroanne C F, Bonjean K, Servotte S, Devy L, Colige A, Clausse N, Blacher S, Verdin E, Foidart J M, Nusgens B V, Castronovo V. (2002) Histone deacetylases inhibitors as anti-angiogenic agents altering vascular endothelial growth factor signaling. Oncogene. 21(3):427-36.
[89] Mineur P, Guignandon A, Lambert ChA, Amblard M, Lapiere ChM, Nusgens B V (2005) RGDS and DGEA-induced [Ca2+]i signalling in human dermal fibroblasts. Biochim Biophys Acta. 1746:28-37.

1. A Vascular Endothelial Growth Factor (VEGF) polypeptide, which polypeptide lacks an amino acid sequence encoded by exon 5 of the VEGF gene.

2. A VEGF polypeptide according to claim 1 which comprises at least the sequence encoded by exon 4 of the VEGF gene.

3. A VEGF polypeptide according to claim 1 which comprises the sequence encoded by exons 3, 4 and 8 of the VEGF gene.

4. A VEGF polypeptide according to claim 1 which comprises the sequence encoded by exons 1 and 2 of the VEGF gene.

5. A VEGF polypeptide according to claim 1 which comprises the sequence as recited in SEQ ID NO:2 or SEQ ID NO:4.

6. A purified nucleic acid molecule which encodes a polypeptide according to claim 1.

7. A purified nucleic acid molecule according to claim 6, which comprises the nucleic acid sequence as recited in SEQ ID NO:1 or SEQ ID NO:3, or is a redundant equivalent or fragment thereof.

8. A vector comprising a nucleic acid molecule as recited in claim 6.

9. A host cell transformed with a vector according to claim 8.

10. A ligand which binds specifically to a VEGF polypeptide according to claim 1.

11. A ligand according to claim 10, which is an antibody or an aptamer.

12. A compound that either increases or decreases the level of expression or activity of a polypeptide according claim 1.

13. A compound according to claim 12 that binds to a polypeptide according to claim 1 without inducing any of the biological effects of the polypeptide.

14. A compound according to claim 13, which is a natural or modified substrate, ligand, enzyme, receptor or structural or functional mimetic.

15. A polypeptide according to claim 1 for use in therapy, therapy monitoring, diagnosis or prognosis of disease.

16. A method of diagnosing a disease in a patient, comprising assessing the level of expression of a natural mRNA encoding a polypeptide according to claim 1, or assessing the activity of a polypeptide according to claim 1, in tissue from said patient and comparing said level of expression or activity to a control level, wherein a level that is different to said control level is indicative of disease.

17. A method according to claim 16 that is carried out in vitro.

18. A method according to claim 16, which comprises the steps of:
(a) contacting a ligand according to claim 10 with a biological sample under conditions suitable for the formation of a ligand-polypeptide complex; and
(b) detecting said complex.

19. A method according to claim 17, comprising the steps of:
a) contacting a sample of tissue from the patient with a nucleic acid probe under stringent conditions that allow the formation of a hybrid complex between a nucleic acid molecule according to claim 6 and the probe;
b) contacting a control sample with said probe under the same conditions used in step a); and
c) detecting the presence of hybrid complexes in said samples; wherein detection of levels of the hybrid complex in the patient sample that differ from levels of the hybrid complex in the control sample is indicative of disease and/or adverse effect of therapy.

20. A method according to claim 16, comprising:
a) contacting a sample of nucleic acid from tissue of the patient with a nucleic acid primer under stringent conditions that allow the formation of a hybrid complex between a nucleic acid molecule according to claim 6 and the primer;
b) contacting a control sample with said primer under the same conditions used in step a); and
c) amplifying the sampled nucleic acid; and
d) detecting the level of amplified nucleic acid from both patient and control samples;
wherein detection of levels of the amplified nucleic acid in the patient sample that differ significantly from levels of the amplified nucleic acid in the control sample is indicative of disease and/or adverse effect of therapy.

21. A method according to claim 16, wherein said disease includes, but is not limited to, cellular trauma, including ulcers, radiation-induced ulcers, any type of wound healing problems; cell proliferative disorders including myeloproliferative disorders such as leukemia, lymphoma, myelodysplastic syndromes and carcinoma; neoplasm, melanoma, lung, colorectal, breast, pancreas, head and neck and other solid tumours; cardiovascular disorders; neurological disorders; diabetes, in particular diabetic blindness, diabetic kidney disease; age-related macular degeneration; rheumatoid arthritis; psoriasis; cerebral and peripheric ischemia; stroke; coronary artery disease; kidney disorders, hemolytic uremic syndrome; developmental disorders, reproductive disorders in particular erectile dysfunction, endometriosis, preeclampsia; and infections.

22. A pharmaceutical composition comprising a polypeptide according to claim 1.

23. A polypeptide according to claim 1, for use in the manufacture of a medicament for the treatment of cellular trauma, including ulcers, radiation-induced ulcers, any type of wound healing problems; cell proliferative disorders including myeloproliferative disorders such as leukemia, lymphoma, myelodysplastic syndromes and carcinoma; neoplasm, melanoma, lung, colorectal, breast, pancreas, head and neck and other solid tumours; cardiovascular disorders; neurological disorders; diabetes, in particular diabetic blindness, diabetic kidney disease; age-related macular degeneration; rheumatoid arthritis; psoriasis; cerebral and peripheric ischemia; stroke; coronary artery disease; kidney disorders, hemolytic uremic syndrome; developmental disorders, reproductive disorders in particular erectile dysfunction, endometriosis, preeclampsia; and infections.

24. A method of treating a disease in a patient, comprising administering to the patient a polypeptide according to claim 1.

25. A method according to claim 24, wherein, for diseases in which the expression of the natural gene or the activity of the polypeptide is lower in a diseased patient when compared to the level of expression or activity in a healthy patient, the polypeptide, nucleic acid molecule, vector, ligand, compound or composition administered to the patient is an agonist.

26. A method according to claim 24, wherein, for diseases in which the expression of the natural gene or activity of the polypeptide is higher in a diseased patient when compared to the level of expression or activity in a healthy patient, the polypeptide, nucleic acid molecule, vector, ligand, compound or composition administered to the patient is an antagonist.

27. A method of monitoring the therapeutic treatment of disease in a patient, comprising monitoring over a period of time the level of expression or activity of a polypeptide according to claim 1, wherein modulating said level of expression or activity over the period of time towards a control level is indicative of regression of said disease.

28. A method for the identification of a compound that is effective in the treatment and/or diagnosis of disease, comprising contacting a polypeptide according to claim 1, with one or more compounds suspected of possessing binding affinity for said polypeptide or nucleic acid molecule, and selecting a compound that binds specifically to said nucleic acid molecule or polypeptide.

29. Use of a polypeptide according to claim 1 for modulating angiogenesis.

30. Use according to claim 29, wherein the modulation of angiogenesis results in an increase in angiogenesis.

31. Use according to claim 30, wherein angiogenesis is increased in muscle tissue;
heart tissue; brain tissue; lung tissue; erectile tissue; hepatic tissue; kidney tissue; placenta or skin.

32. Use according to claim 30, wherein angiogenesis is increased at a site of cellular trauma.

33. Use according to claim 32, wherein the site of cellular trauma is in muscle tissue;
heart tissue; brain tissue; lung tissue; erectile tissue; hepatic tissue; kidney tissue; placenta or skin.

34. Use according to claim 33, wherein the cellular trauma is in the form of a non-healing wound.

35. Use according to claim 29, wherein the modulation of angiogenesis results in a decrease or inhibition of angiogenesis.

36. Use according to claim 29, wherein the decrease or inhibition of angiogenesis is in tissue wherein increased activity of VEGF is causing a disease state.

37. Use according to claim 36, wherein the disease state is cancer, rheumatoid arthritis, psoriasis or angiogenic diseases of the eye.

38. Use according to any of claims 29 wherein said medicament is administered orally, intravenously, intramuscularly, intra-arterially, intramedullary, intrathecally, intraventricularly, transdermally, subcutaneously, intraperitoneally, intranasally, enterally, topically, sublingually, intravaginally or rectally.

39. A method for inducing or increasing the expression of a VEGFΔ5 polypeptide, comprising exposing tissues capable of expressing a VEGFΔ5 polypeptide to UV-B radiation.

40. The method of claim 39 for the treatment of chronic wounds, in particular chronic ulcers.

41. A kit useful for diagnosing disease comprising a first container containing a nucleic acid probe that hybridises under stringent conditions with a nucleic acid molecule according to claim 6; a second container containing primers useful for amplifying said nucleic acid molecule; and instructions for using the probe and primers for facilitating the diagnosis of disease.

42. The kit of claim 41, further comprising a third container holding an agent for digesting unhybridised RNA.

43. A kit comprising an array of nucleic acid molecules, at least one of which is a nucleic acid molecule according to claim 6.

44. A kit comprising one or more antibodies or aptamers, at least one of which bind to a polypeptide as recited in claim 1; and a reagent useful for the detection of a binding reaction between said antibody and said polypeptide.

45. A transgenic or knockout non-human animal that has been transformed to express higher, lower or absent levels of a polypeptide according to claim 1.

46. A method for screening for a compound effective to treat disease, by contacting a non-human transgenic animal according to claim 45 with a candidate compound and determining the effect of the compound on the disease of the animal.

47. Method of selecting biologically active compounds comprising:
(i) contacting a candidate compound with recombinant host cells expressing a VEGFΔ5 polypeptide;
(ii) selecting compounds that bind said VEGFΔ5 polypeptide and/or that modulate the activity of the VEGFΔ5 polypeptide.

48. A kit comprising a peptide of claim 1.
